(19) United States
(12) Patent Application Publication
(10) Pub. No.: US 2022/0288128 A1
(43) Pub. Date: Sep. 15, 2022

(54) MESENCHYMAL STEM CELL COMPOSITIONS

(71) Applicant: Akan Biosciences LLC, Germantown, MD (US)

(72) Inventor: Mukesh Kumar, Germantown, MD (US)

(21) Appl. No.: 17/627,542

(22) PCT Filed: Jul. 16, 2020
(86) PCT No.: PCT/US2020/042339

§ 371 Date: Jan. 14, 2022

Related U.S. Application Data
(60) Provisional application No. 62/874,672, filed on Jul. 16, 2019.

(51) Int. Cl. A61K 35/28; C12N 5/0775; A61K 35/35

(52) U.S. Cl. CPC A61K 35/28; C12N 5/0667; A61K 35/35

(57) ABSTRACT

The present disclosure relates to compositions that comprise an enriched stromal vascular fraction, and methods of use thereof.

### FIELD OF THE DISCLOSURE

The present disclosure relates to compositions that comprise an enriched stromal vascular fraction, and methods of use thereof.

### BACKGROUND OF THE DISCLOSURE

Despite the excitement surrounding stem cell therapy, there is still a significant rate of therapeutic failures — not every patient responds to stem cell therapy. There are many factors that explain why stem cell procedures may not be successful in certain patient populations. For instance, stem cell properties such as absolute number, plasticity, senescence, marker expression, proliferation, and trophic and differentiation activities, decline in elderly and obese subjects. Using these functionally impaired stem cells, then, in an autologous stem cell treatment is unlikely to produce significant clinical benefit. This is a critical limitation for current autologous stem cell therapy.

In particular, the potential for impaired functionality of autologous stem cells is a concern for osteoarthritis focused treatments, as patients seeking treatment for osteoarthritis are more likely to suffer from obesity and be older. Similarly, autologous stem cells from diabetic patients exhibit altered intrinsic properties such as marker expression, inadequate migration and impaired differentiation. Furthermore, stem cells from diabetic patients do not secrete an adequate complement of trophic factors required for triggering matrix repair, regeneration and anti-inflammatory effects. Given that people with diabetes are twice as likely to develop arthritis, the limitation of the autologous approach constitutes a significant barrier to this group of patients.

Almost all of the stem cell based therapies in development or in unapproved clinics are autologous in nature and involve harvesting the stem cells and injecting them into the damaged area of the same subject during the same surgery. One consequence of these same day procedures is that the clinics do not have sufficient time to characterize their stem cell product before transplantation into a patient. Thus, the stem cell quality is not fully ascertained, and the stem cell populations are not fully characterized before using the cells for clinical applications. This ambiguity presents issues for fully exploiting the potential benefit of stem cells.

Hence, there is a need in the art for a stem cell composition that is sourced from suitable donors that can be used for allogeneic stem cell treatments.

### BRIEF DESCRIPTION OF THE FIGURES

FIG. 1 depicts plots illustrating the immunophenotyping of MSCs: An aliquot of ASCs purified and stained against CD90, CD166 and CD29. Data were analyzed by FlowJo. Unstained samples, and control samples with green, yellow and red dyes served as background control (not shown). Approximately 85% of the cells expressed the MSC marker CD90.

FIG. 2A-LL depicts FACS plots of MUSE quality control data on indicated days.

### DETAILED DESCRIPTION

The present disclosure relates to compositions and methods of use thereof. Specifically, the present disclosure encompasses a composition comprising an enriched stromal vascular fraction, and methods of use for such a fraction. Generally speaking, an enriched stromal vascular fraction of the disclosure may be used for allogeneic stem cell treatments. Such compositions and methods are described in further detail below.

### I. Composition

One aspect of the present disclosure is a composition comprising an enriched stromal vascular fraction. As used herein, the phrase “stromal vascular fraction” refers to an aqueous fraction of an adipose tissue sample that contains nucleated cells, such as adipose derived stem cells. In some embodiments, a stromal vascular fraction may further comprise additional nucleated cell types, such as endothelial precursor cells (EPCs), smooth muscle cells, pericytes, and pre-adipocytes among others. Generally speaking, however, such additional cell types comprise less than about 15% of the total cells in the enriched stromal vascular fraction of the present disclosure. For example, in certain embodiments, an enriched stromal vascular fraction of the present disclosure comprises less than about 15%, less than about 14%, less than about 13%, less than about 12%, less than about 11%, less than about 10%, less than about 9%, less than about 8%, less than about 7%, less than about 6%, or less than about 5% of cells other than adipose derived stem cells.

The phrase “adipose derived stem cells” refers to mesenchymal stem cells that originate from the stromal fraction of adipose tissue. In preferred embodiments, an enriched stromal vascular fraction of the present disclosure is enriched in adipose derived stem cells. As used herein, the term “enriched” means that a stromal vascular fraction of the present disclosure comprises at least about 85% viable adipose derived stem cells. This percentage is greater than any other known stromal vascular fraction preparation.

Mesenchymal stem cells (MSCs) are non-hematopoietic stem cells that are able to differentiate into mesenchymal tissues such as bone, cartilage, muscle, ligament, tendon, organ, tissue, neuron, cardiocytes, pancreatic cells, and other tissues. Adipose derived mesenchymal stem cells are isolated from adipose tissue. Adipose derived mesenchymal stem cells may be characterized by cell surface marker expression and cell yield/viability.

An enriched stromal vascular fraction of the present disclosure is derived from adipose tissue collected from a subject. As used herein, adipose tissue refers to any viable fat tissue. In certain embodiments, the adipose tissue is visceral fat tissue. Such tissue may be comprised of ex vivo cells collected from a subject, primary cells in culture that were derived from a subject, or an immortalized cell line derived from a subject.

Adipose tissue of the present disclosure may be collected from any organism having fat tissue. Typically, the adipose tissue is collected from a mammal. For instance, the adipose tissue may be collected from a rodent, such as mice or rats. In other embodiments, the adipose tissue may be collected from non-human primates. In still other embodiments, the adipose tissue may be collected from a human. Generally speaking, the gender of the human is immaterial to the present disclosure. In some embodiments, the adipose tissue is a lipoaspirate from a lipoaspirate surgery (such as liposuction). The means of obtaining the adipose tissue from the subject, however, is not critical to the invention.

In embodiments where the adipose tissue is a lipoaspirate, an adipose tissue stromal vascular fraction may be derived from a tumescent fraction of a lipoaspirate. In other embodiments where the adipose tissue is a lipoaspirate, an adipose tissue stromal vascular fraction may be derived from a fat fraction of a lipoaspirate. Means of collecting tumescent fractions and fat fractions of a lipoaspirate are known in the art.

Importantly, the adipose tissue collected from a subject must meet one or more criteria. For instance, in one embodiment, the adipose tissue is collected from a subject between about 5 and about 45 years of age. In preferred embodiments, the subject is between about 18 and about 45 years of age. In another embodiment, the adipose tissue is collected from a visceral fat source. In yet another embodiment, the adipose tissue is collected from a subject with a body mass index (BMI) of less than 30. In some embodiments, the adipose tissue is collected from a subject between 18 and 45 years of age, from a visceral fat source, and from a subject with a BMI of less than 30.

In a further embodiment, the adipose tissue is collected from a subject with no clinically significant abnormalities in a physical exam administered within 6 months prior to taking the adipose tissue, preferably 3 months. In another further embodiment, the adipose tissue is collected from a subject with no lipoma or lipomatosis. In yet another further embodiment, the adipose tissue is collected from a subject with no known active infectious disease. In still another embodiment, the adipose tissue is collected from a subject with a systolic blood pressure (supine) between about 90 mmHg and about 180 mmHg. In yet another embodiment, the adipose tissue is collected from a subject with a resting heart rate less than about 99 bpm. In additional embodiments, the adipose tissue is collected from a subject with no known history of lipoma/lipomatosis, no known history of cancer in the last five years, no known history of lymphoma, leukemia or Kaposi sarcoma, and no known history of diabetes.

In one embodiment, adipose tissue is collected from a subject between 18 and 45 years of age, from a visceral fat source, from a subject with a BMI of less than 30, from a subject with no clinically significant abnormalities in the physical exam, from a subject with no lipoma or lipomatosis, from a subject with no known active infectious disease, from a subject with a systolic blood pressure (supine) between about 90 mmHg and about 180 mmHg, from a subject with a resting heart rate less than about 99 bpm, and from a subject with no known history of lipoma/lipomatosis, no known history of cancer in the last five years, no known history of lymphoma, leukemia or Kaposi sarcoma, and no known history of diabetes.

In each of the above embodiments, a subject may be assigned a unique Donor ID (DID). The DID may be linked to the subject's screening information. As used herein, screening information refers to any information used to determine the suitability of the subject as a source of adipose tissue.

An enriched stromal vascular fraction of the present disclosure comprises about 4.2×10⁶ to about 9.2×10⁷ nucleated cells per 100 ml of adipose tissue. In some embodiments, an enriched stromal vascular fraction comprises about 4.2×10⁷ to about 9.2×10⁷ nucleated cells per 100 ml of adipose tissue collected from a subject. For instance, an enriched stromal vascular fraction may comprise about 4.2, 4.3, 4.4, 4.5, 4.6, 4.7, 4.8, 4.9, 5.0, 5.1, 5.2, 5.3, 5.4 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0×10⁶ nucleated cells per 100 ml of adipose tissue collected from a subject, or about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, or 9.2×10⁷ nucleated cells per 100 ml of adipose tissue collected from a subject.

In some embodiments, at least about 75% of the nucleated cells express CD90 on their cell surface. For instance, at least about 75, 76, 77, 78, 79, 80, 81, 82, 83, 84, or 85% of the nucleated cells in the enriched stromal vascular fraction express CD90 on their cell surface. CD90 (Cluster of Differentiation 90; also known as Thy-1) is a 25-37 kDa heavily N-glycosylated, glycophosphatidylinositol (GPI) anchored conserved cell surface protein with a single V-like immunoglobulin domain. Methods of detecting CD90 on the surface of a cell are known in the art. In preferred embodiments, at least about 85% of the nucleated cells express CD90 on their cell surface.

In certain embodiments, at least about 65% of the nucleated cells in the enriched vascular stromal fraction that express CD90 on the cell surface also express CD29. For instance, at least about 65, 66, 67, 68, 69 or 70% of the nucleated cells in the enriched vascular stromal fraction that express CD90 on the cell surface also express CD29. CD29 is also known as Integrin beta-1 (ITGB1), which is a cell surface receptor. In preferred embodiments, at least about 70% of the nucleated cells in the enriched vascular stromal fraction that express CD90 on the cell surface also express CD29.

In some embodiments, only about 5% of the nucleated cells in the enriched vascular stromal fraction express CD34. For instance, only about 5%, 4%, 3%, 2%, 1% or less than 1% of the nucleated cells in the enriched vascular stromal fraction express CD34. CD34 (cluster of differentiation 34) is a transmembrane phosphoglycoprotein protein. In preferred embodiments, less than 1% of the nucleated cells in the enriched vascular stromal fraction express CD34.

In particular embodiments, the nucleated cells do not express detectable levels of CD45 by FACS analysis. CD45 (cluster of differentiation 45) is also referred to as PTPRC (Protein tyrosine phosphatase, receptor type, C) or LCA (leukocyte common antigen). Similarly, in some embodiments, the nucleated cells do not express detectable levels of CD31 by FACS analysis. CD31 (cluster of differentiation 31) is also referred to as Platelet endothelial cell adhesion molecule (PECAM-1).

In preferred embodiments, 85% of the nucleated cells of the enriched stromal vascular fraction express CD90 on their cell surface, of those 85%, 70% express CD29, less than 1% of the nucleated cells express CD34, and the nucleated cells do not express detectable levels of CD45 or CD31.

An enriched stromal vascular fraction of the present disclosure comprises adipose derived mesenchymal stem cells, wherein the adipose derived mesenchymal stem cells are about 95 to about 99% viable, when measured within two hours of completing the enrichment protocol. For instance, the adipose derived mesenchymal stem cells may be about 95, 96, 97, 98, or 99% viable, when measured within two hours of completing the enrichment protocol.

In certain embodiments, an enriched stromal vascular fraction of the present disclosure comprises adipose derived mesenchymal stem cells, wherein the adipose derived mesenchymal stem cells are about 95 to about 99% viable, when measured within twenty-four hours of completing the enrichment protocol. In some embodiments, an enriched stromal vascular fraction of the present disclosure comprises adipose derived mesenchymal stem cells, wherein the adipose derived mesenchymal stem cells are about 95 to about 99% viable, when measured within forty-eight hours of completing the enrichment protocol.

In some embodiments, an enriched stromal vascular fraction of the present disclosure comprises adipose derived mesenchymal stem cells that do not display detectable culture-associated cellular changes.

In some embodiments, an enriched stromal vascular fraction of the present disclosure is stored in one or more vials. For instance, cryovials may be used. In certain embodiments, a vial may hold between about 1 mL and 3 mL. In other embodiments, a vial may hold more than 3 mL. In specific embodiments, a vial may hold at least about 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mL. A vial may hold between about 3×10⁶ viable cells per mL to about 10×10⁶ viable cells per mL. In some embodiments, a vial may hold about 2, 2.1, 2.2, 2.3, 2.4, 2.5, 2.6, 2.7, 2.8, 2.9. 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 ×10⁶ viable cells per mL.

In particular embodiments, a composition comprising an enriched stromal vascular fraction may be filtered before being stored in a vial. In such embodiments, the composition may be referred to as a filtered suspension. In such embodiments, a composition may be filtered with a mesh size of at least 10, 20, 30, 40, 50, 60, 70, 80, 90, or 100 microns.

In particular embodiments, a composition of the present invention that comprises an enriched stromal vascular fraction may further comprise additional components, such as salts, buffers, cryopreservatives, or solvents. In some embodiments, a composition comprising an enriched stromal vascular fraction may further comprise a chloride salt, such as but not limited to, NH₄Cl. In some embodiments, a composition comprising an enriched stromal vascular fraction may further comprise one or more buffers, such as phosphate buffered saline (PBS). In some embodiments, a composition comprising an enriched stromal vascular fraction may further comprise a cryopreservative, also referred to as a freezing solution. In some embodiments, a composition comprising an enriched stromal vascular fraction may further comprise a solvent, such as but not limited to isopropanol. The compositions of the present invention may be stored at room temperature, about zero degrees Celsius, about −20 degrees Celsius, or about −80 degrees Celsius. The total cell viability of the present invention and compositions described herein may continue to be at least 60, 65, 70, 75, 80, 85, 90, 95, or greater than 95% viability after storage for at least 3 months, 6 months, 9 months, 12 months, 18 months, or 24 months when stored at room temperature, zero degrees Celsius, about −20 degrees Celsius, or about −80 degrees Celsius.

### II. Methods of Making a Composition of the Disclosure

One aspect of the present disclosure encompasses methods of making a composition as described in Section I above. In each of these embodiments, the first step is collecting adipose tissue from a subject. Adipose tissue of the present disclosure may be collected from any subject having fat tissue. Typically, the adipose tissue is collected from a mammal. For instance, the adipose tissue may be collected from a rodent, such as mice or rats. In other embodiments, the adipose tissue may be collected from non-human primates. In still other embodiments, the adipose tissue may be collected from a human. Generally speaking, the gender of the human is immaterial to the present disclosure. In some embodiments, the adipose tissue is a lipoaspirate from a lipoaspirate surgery (such as liposuction). The means of obtaining the adipose tissue from the subject, however, is not critical to the invention.

Importantly, the adipose tissue collected from a subject must meet one or more criteria. For instance, in one embodiment, the adipose tissue is collected from a subject between about 5 and about 45 years of age, preferably between about 18 and about 45 years of age. In another embodiment, the adipose tissue is collected from a visceral fat source. In yet another embodiment, the adipose tissue is collected from a subject with a BMI of less than 30. In some embodiments, the adipose tissue is collected from a subject between 18 and 45 years of age, from a visceral fat source, and from a subject with a BMI of less than 30.

In a further embodiment, the adipose tissue is collected from a subject with no clinically significant abnormalities in a physical exam administered within 6 months prior to taking the adipose tissue, preferably 3 months. In another further embodiment, the adipose tissue is collected from a subject with no lipoma or lipomatosis. In yet another further embodiment, the adipose tissue is collected from a subject with no known active infectious disease. In still another embodiment, the adipose tissue is collected from a subject with a systolic blood pressure (supine) between about 90 mmHg and about 180 mmHg. In yet another embodiment, the adipose tissue is collected from a subject with a resting heart rate less than about 99 bpm. In additional embodiments, the adipose tissue is collected from a subject with no known history of lipoma/lipomatosis, no known history of cancer in the last five years, no known history of lymphoma, leukemia or Kaposi sarcoma, and no known history of diabetes.

In one embodiment, adipose tissue is collected from a subject between 18 and 45 years of age, from a visceral fat source, from a subject with a BMI of less than 30, from a subject with no clinically significant abnormalities in the physical exam, from a subject with no lipoma or lipomatosis, from a subject with no known active infectious disease, from a subject with a systolic blood pressure (supine) between about 90 mmHg and about 180 mmHg, from a subject with a resting heart rate less than about 99 bpm, and from a subject with no known history of lipoma/lipomatosis, no known history of cancer in the last five years, no known history of lymphoma, leukemia or Kaposi sarcoma, and no known history of diabetes.

In each of the above embodiments, a subject may be assigned a unique Donor ID (DID). The DID may be linked to the subject's screening information.

As used herein, screening information refers to any information used to determine the suitability of the subject as a source of adipose tissue.

After collection of adipose tissue, the adipose tissue is treated to create the enriched stromal vascular fraction. Generally speaking, an enriched stromal vascular fraction can be created from about a 100 ml adipose tissue sample. In some embodiments, an enriched stromal vascular fraction can be created from about a 100, 150, 200, 250, 300, 350, 400, 450, 500, 550, 600, 650, 700, or 750 ml adipose tissue sample. Initially, a stromal vascular fraction may be isolated by using mechanical treatment, not enzymatic treatment. Specifically, the adipose tissue may be treated with ultrasonic waves. For example, an adipose tissue may be exposed to ultrasonic waves using a suitable probe for less than 15 minutes at about 20 to about 30 kHz. The goal of the ultrasonication is to lyse any adipose cells or break-up blood vessel cells in the adipose tissue, thereby dissociating or releasing substantial numbers of intact stromal vascular fraction cells from the lysed blood vessels while substantially maintaining the viability of the cells constituting the stromal vascular fraction. In another embodiment, the ultrasonication may be effected for less than about 8 minutes, paused, and then continued again for less than about 8 minutes for a total time of less than 15 minutes. In another embodiment, the probe may be placed towards the bottom of the tissue sample for a first ultrasonication period, paused, and then moved half-way up the tissue sample and continued for a second ultrasonication period. In certain embodiments, there may be two, three, four or more than four ultrasonication periods. In each of the above embodiments, the ultrasonic waves may be at a frequency of about 20-30 kHz, optionally about 20, 21, 22, 23, 24, 24, or 25 kHz, or optionally about 20-23 kHz or 23-25 kHz. In another embodiment, the ultrasonication may be performed using an ultrasonic probe of about 10-15 mm, for instance, about 10, 11, 12, 13, or 14 mm probe. In a specific embodiment, a 14 mm probe may be used. In another embodiment, the ultrasonication device may have a 200W-500W generator.

Before or after ultrasonication, the cells may be centrifuged, for example at about 1,000 RPM, about 2,000 RPM, about 3,000 RPM, about 4,000 RPM, or about 5,000 RPM for about 1 to about 3 minutes, about 3 to about 5 minutes, about 5 to about 7 minutes, or about 7 to about 10 minutes. Following ultrasonication and/or centrifugation, the isolated cells or pellet may be suspended in appropriate volume of media for desired cell concentration.

In a method of the present invention, cell viability may be tested at several different points in the method. At each tested point, the total cell viability should be at least 60%. For instance, the total cell viability may be at least 60, 65, 70, 75, 80, 85, 90, 95, or greater than 95% viability. In particular embodiments, total cell viability may be measured before the adipose tissue is -+processed (e.g. the raw adipose tissue), before filtering, after filtering, before cryopreservation, or after cryopreservation.

III. Methods of Use

Another aspect of the present invention is use of a composition as described in section I above as a therapy for a subject in need of such therapy. Non-limiting examples of subjects in need of such therapy may include subjects in need of treatment for gum recession, loss of bone (including the jaw), amyotrophic lateral sclerosis (ALS), osteoarthritis, rheumatoid arthritis, autism, diabetes (including Type I diabetes), bone fractures, chronic obstructive pulmonary disease (COPD), burns and non-healing wounds, enterocutaneous fistula (HULPUTC), gingival gum regeneration, hair loss, ischemic heart failure, microvascular protection treatment in a myocardial infarction, migraine, multiple sclerosis, orthopedic problems, plantar fasciitis, recto-vaginal fistula, rotator cuff injuries, sports injuries, optionally tears and sprains of the ligaments and tendons, tennis elbow, tinnitus, and ulcers.

Importantly, in some embodiments, a composition of the present disclosure may be used to treat a subject directly, without the need for culturing the cells in the composition.

### EXAMPLES

The Examples detailed herein are intended to demonstrate a specific embodiment of the present disclosure, and are not to be taken as limiting. One of skill in the art will appreciate that variations can and will fall within the scope of this disclosure.

### Example 1—Identify Suitable Donors

Since the quality of stem cells is a major determinant of clinical efficacy, the ideal stem cell donor traits were determined. In particular, it was determined that three critical attributes determined the quality and consistency of an enriched stromal vascular fraction when the adipose tissue collection method and biomanufacturing process are held constant. These are: fat depot source, the donor age, and BMI. An enriched stromal vascular fraction derived from the visceral fat depot of young subjects (<45 yrs) with a BMI <30 provided the highest quality and most consistent enriched stromal vascular fractions, as determined by yield of nucleated cells, viability, and adipose derived mesenchymal stem cell marker expression.

Based on these findings, the following criteria for donor eligibility were developed: 1) subjects aged between 18-45 years, 2) visceral fat source, 3) BMI <30, 4) no clinically significant abnormalities in a physical exam performed within 6 months prior to the adipose tissue collection, and 5) no lipoma or lipomatosis. Donors with the following criteria were excluded: 1) Active infectious disease, 2) Systolic BP (supine) ≥90 mmHg or ≥180 mmHg, c) Resting HR ≥100 bpm, 3) History of lipoma/lipomatosis 4) History of cancer in the last five years, 5) History of lymphoma, leukemia or Kaposi sarcoma, or 6) History of diabetes.

### Example 2: Enriched Stromal Vascular Fraction Marker Characterization

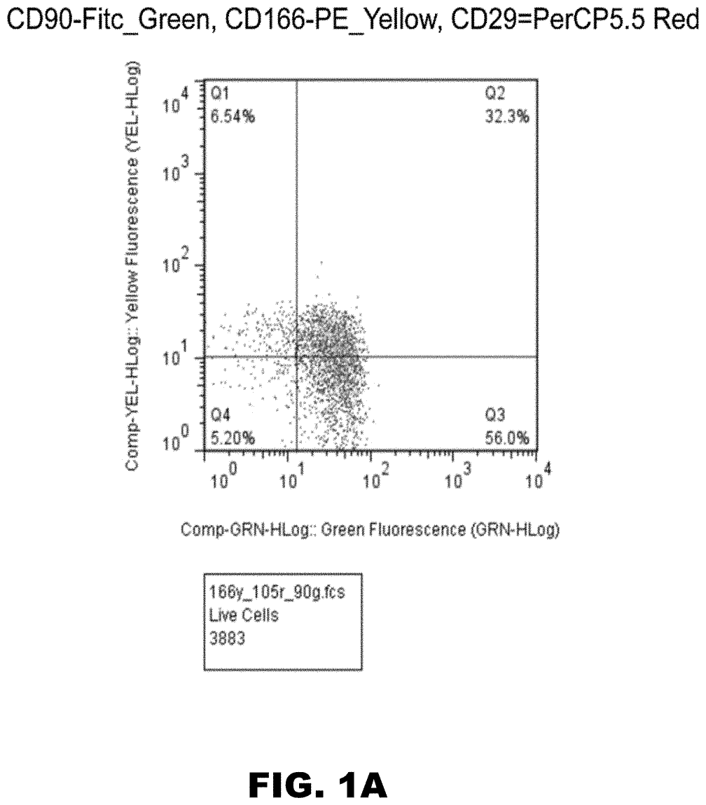

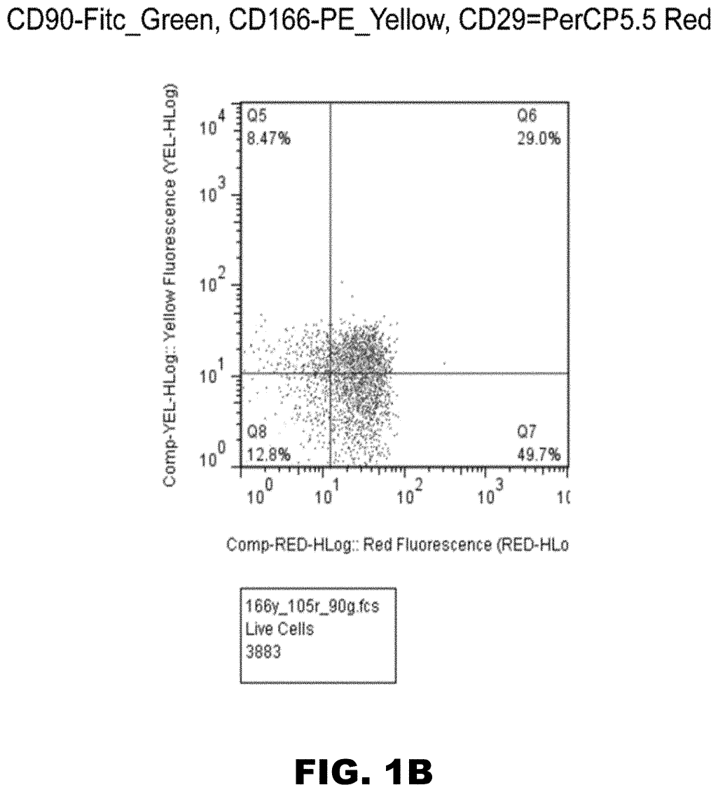

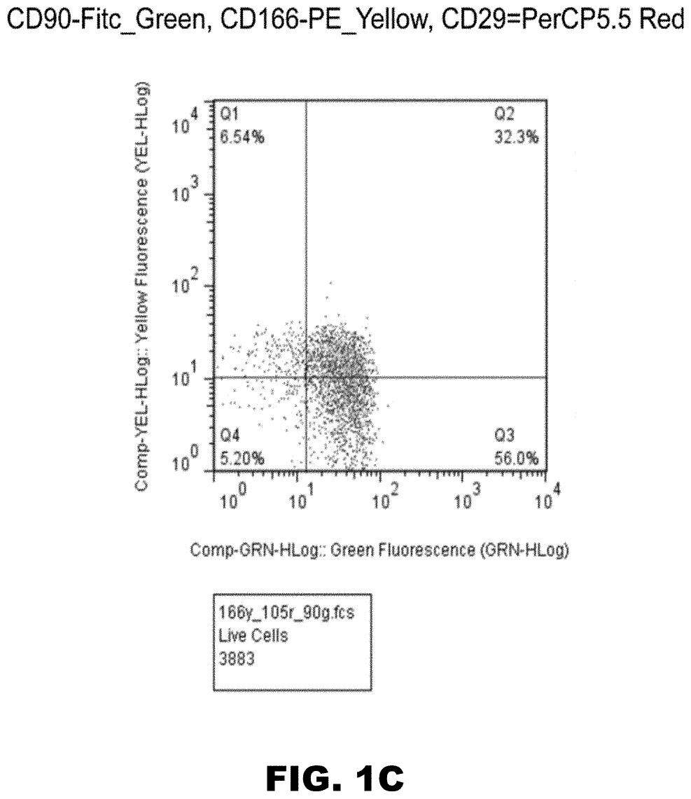

Eighty-five percent of the enriched stromal vascular fraction nucleated cells express the mesenchymal stem cell marker CD90 (FIG. 1). Seventy percent of the cells expressing CD90 also expressed CD29, and 30% expressed CD166. CD34 expression was detected in less than 1% of cells in the enriched stromal vascular fraction.

Enriched stromal vascular fraction cells were negative for CD45 (a B and T cell marker) and CD31 (a marker for endothelial cells, platelets, macrophages, NK cells, lymphocytes, neutrophils and osteoclasts). These data indicate that nucleated cells from the enriched stromal vascular fraction express mesenchymal stem cell surface markers.

Unlike the traditional purification method, an enriched stromal vascular fraction of the present disclosure may be harvested from both the tumescent fraction and the fat fraction. The enriched stromal vascular fraction produced by this method consistently yields about 4.2 to about 9.2×10⁷ nucleated cells/100 ml raw material, with 95-99% viability. Because of this high yield, an enriched stromal vascular fraction of the present disclosure does not require in vitro culture and expansion before use in vivo. This avoids culture-associated cellular changes, reducing both contamination risk and costs.

### Example 3: Quality Control Data

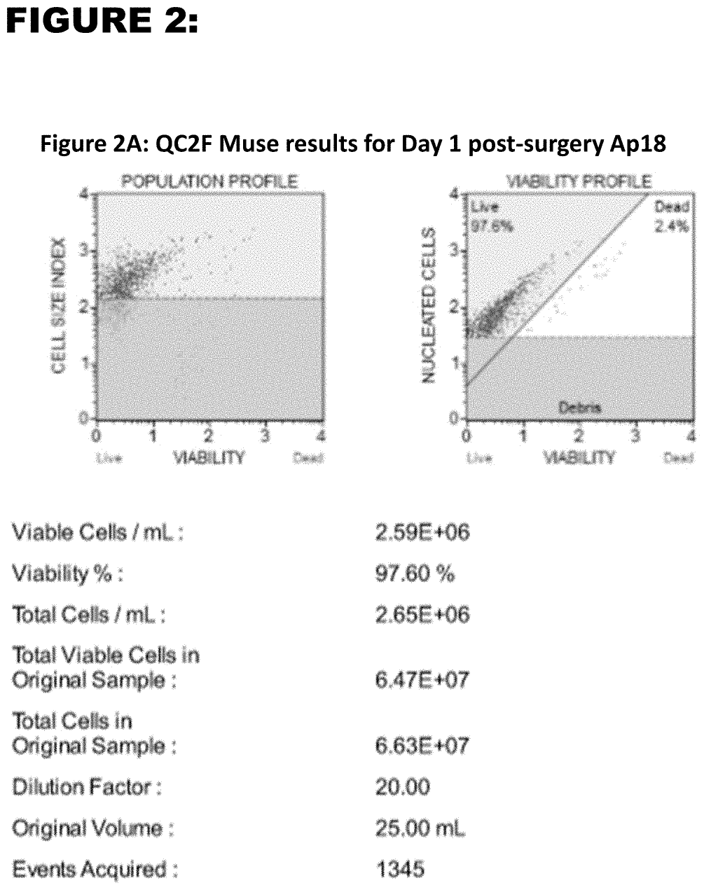

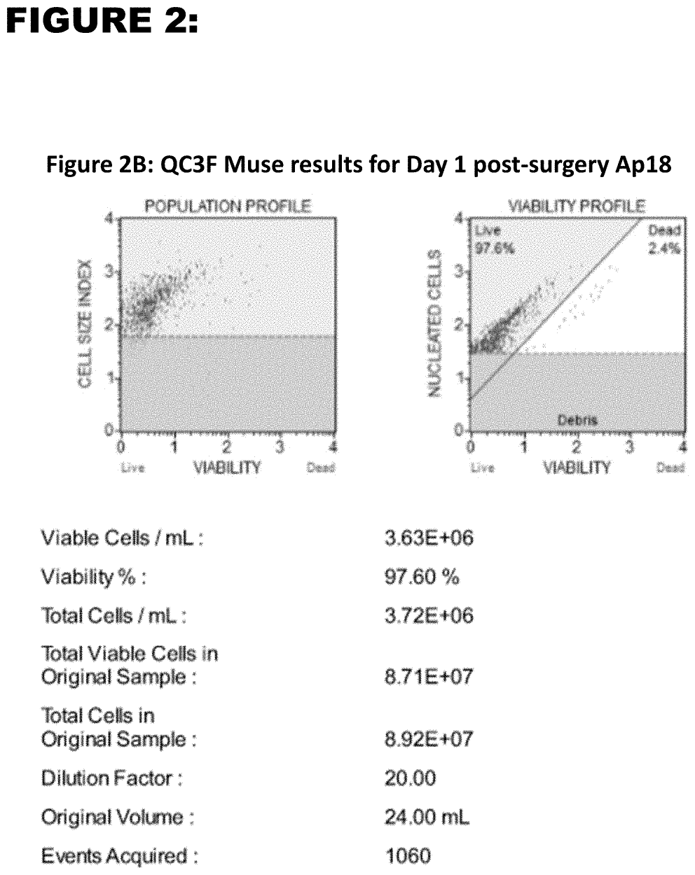

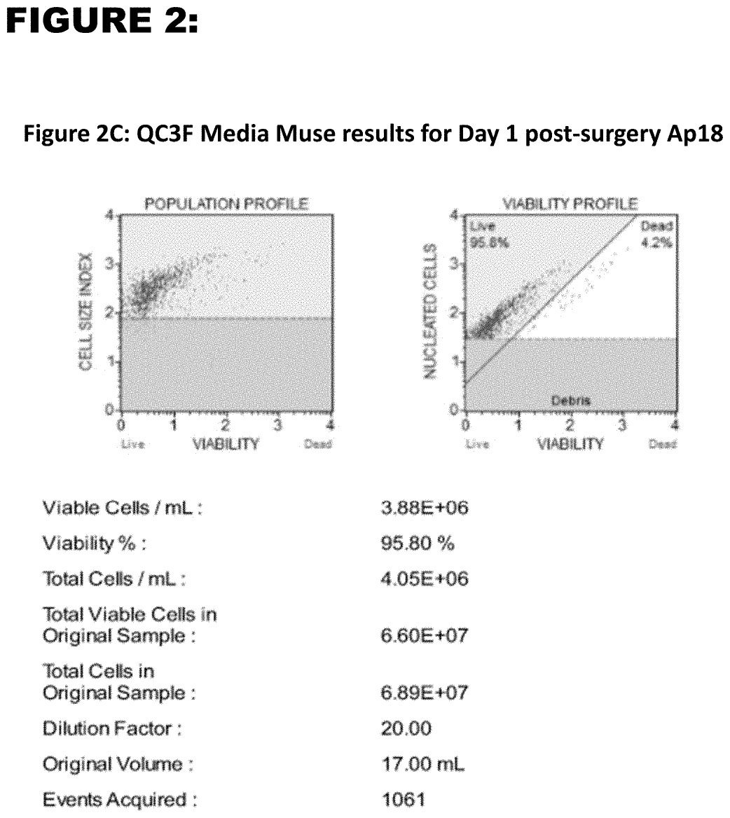

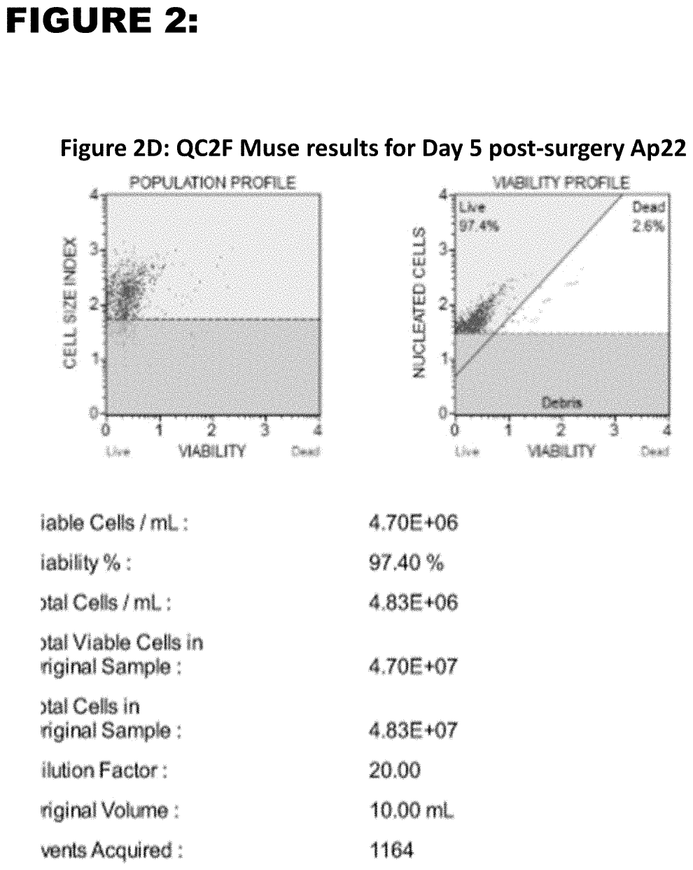

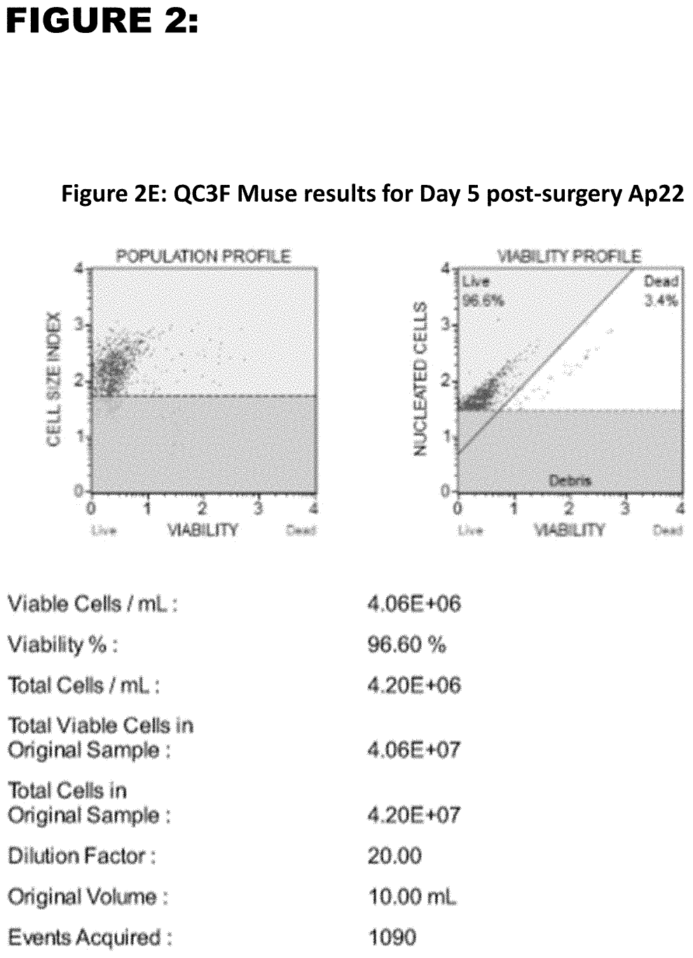

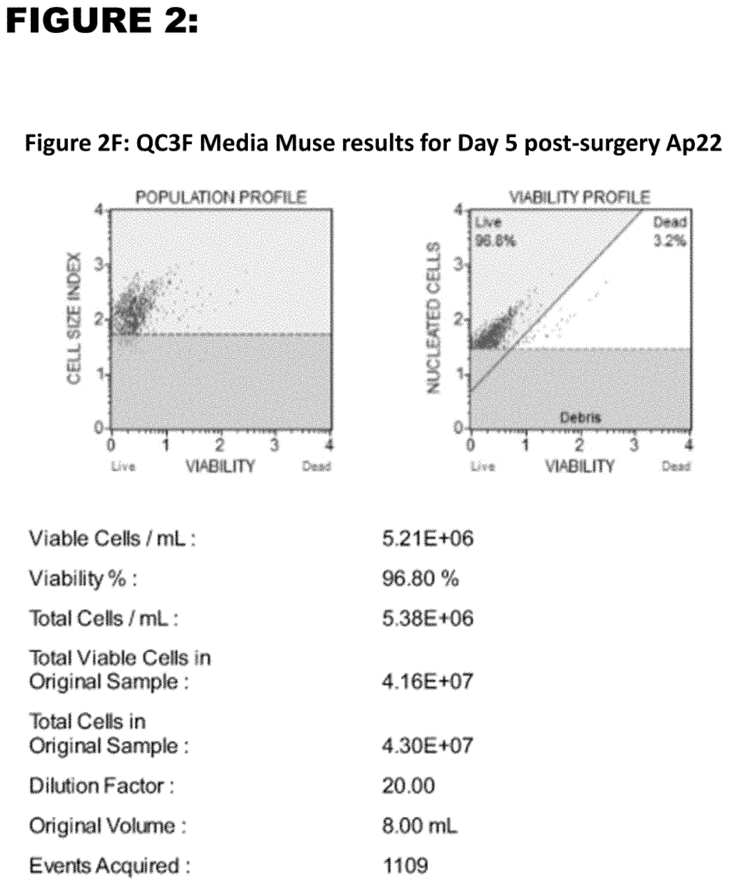

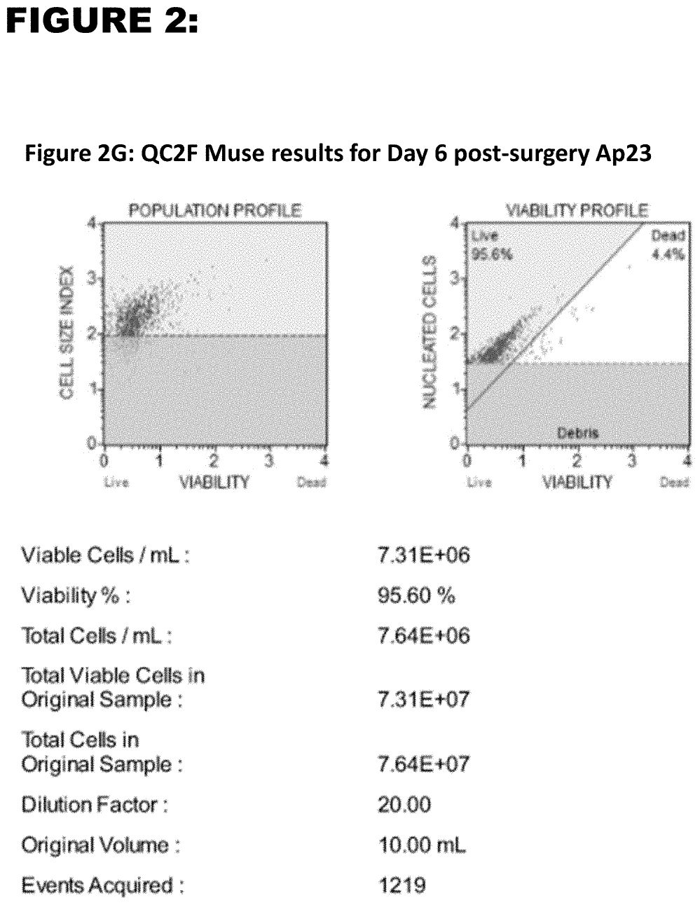

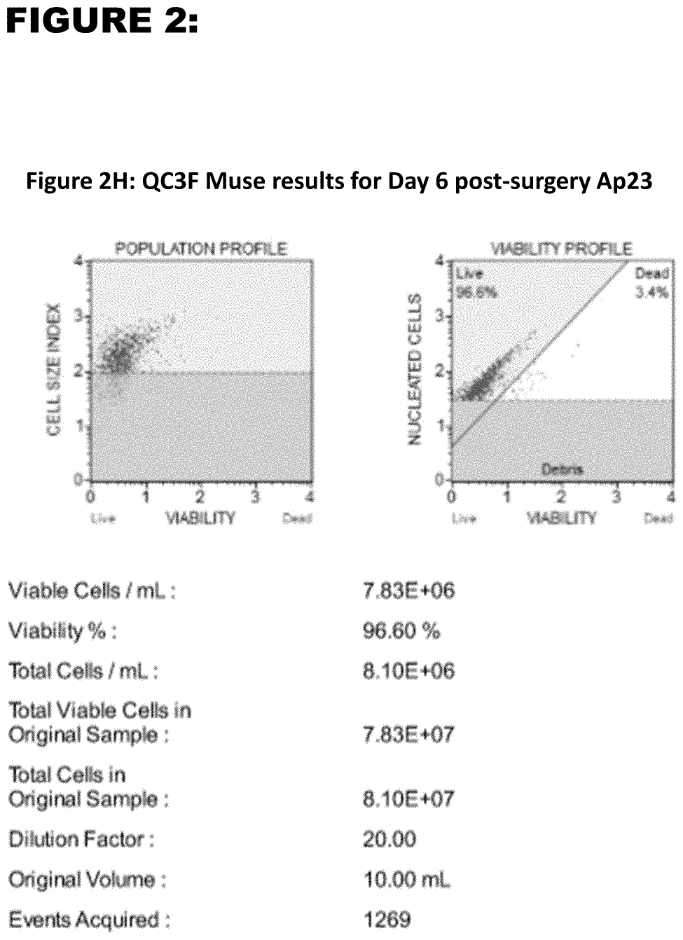

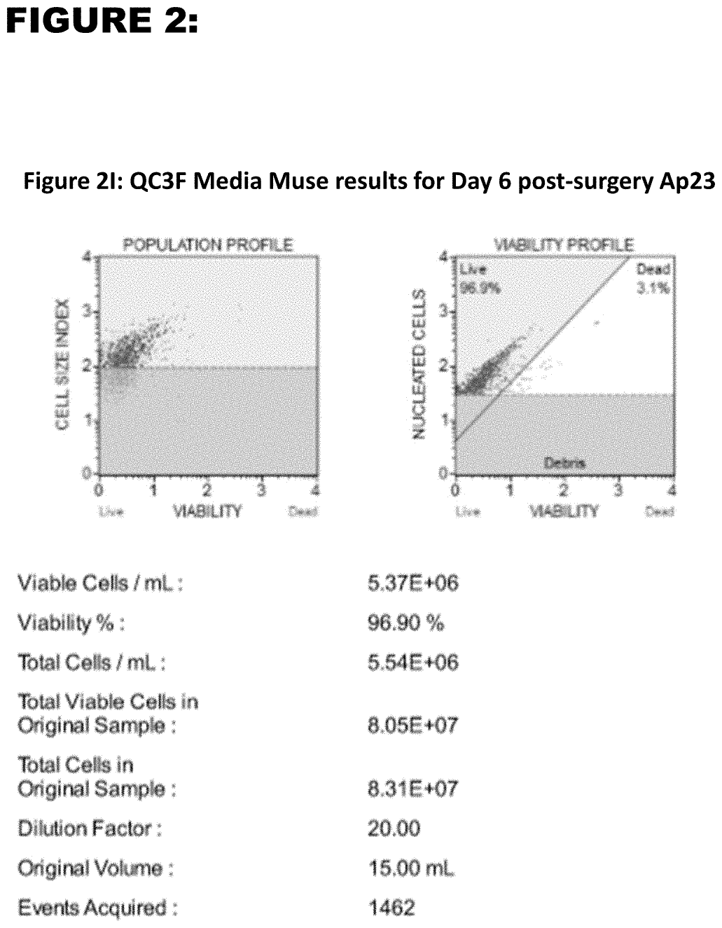

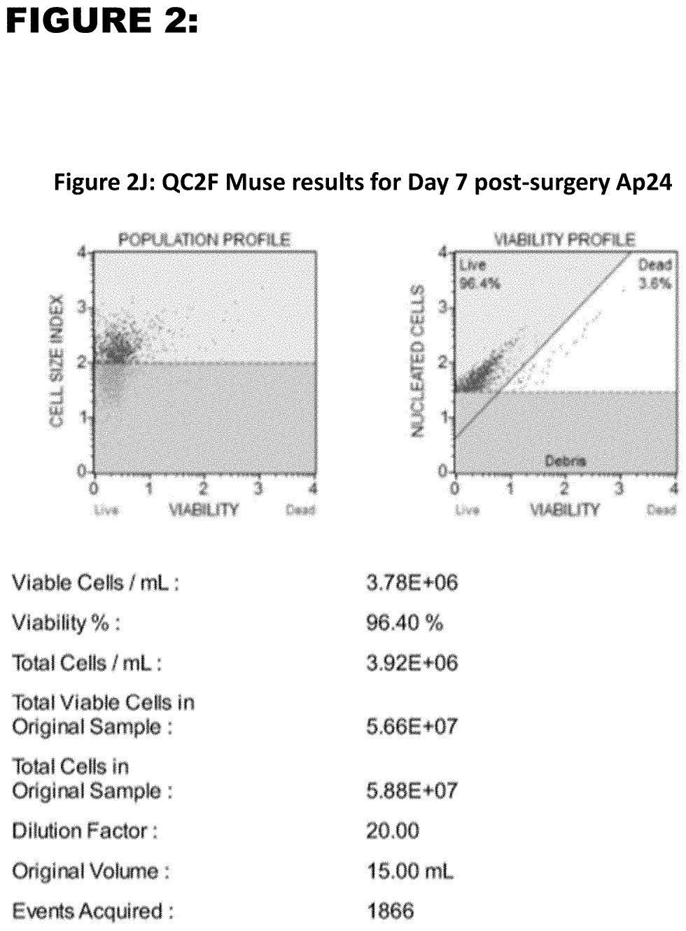

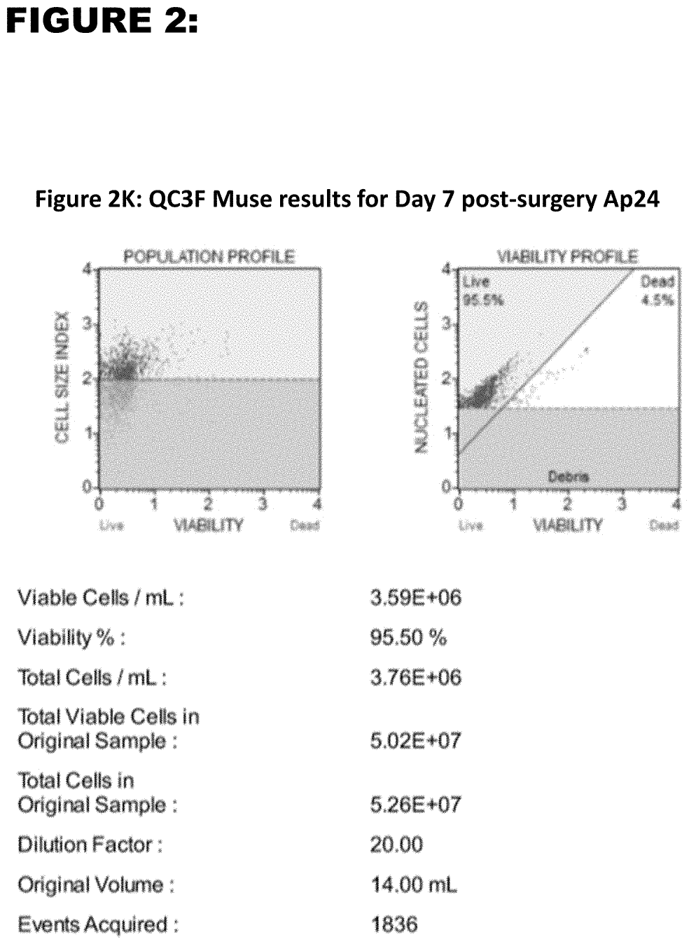

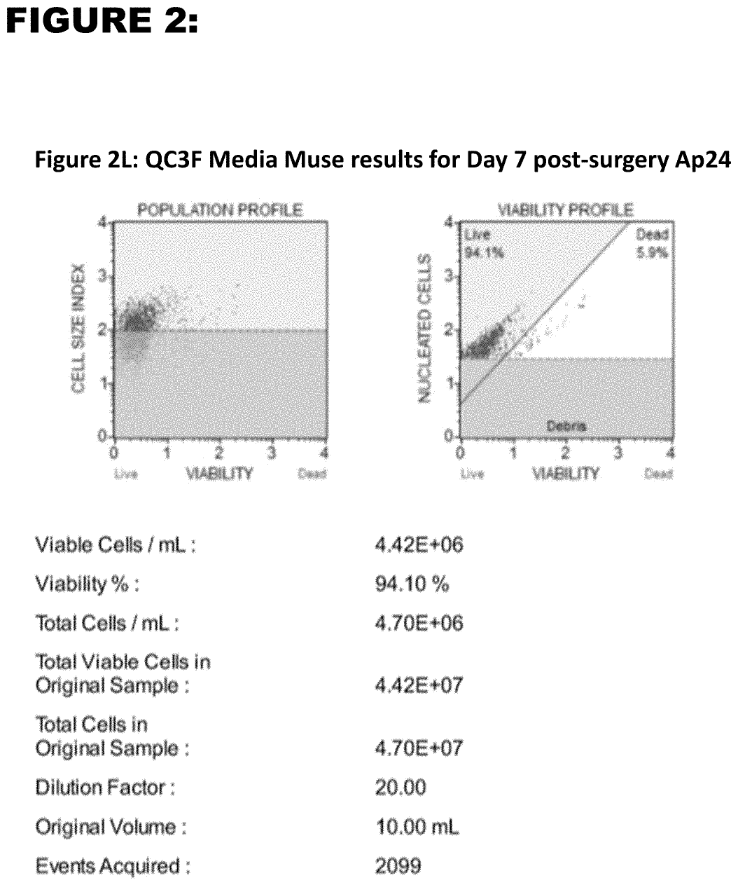

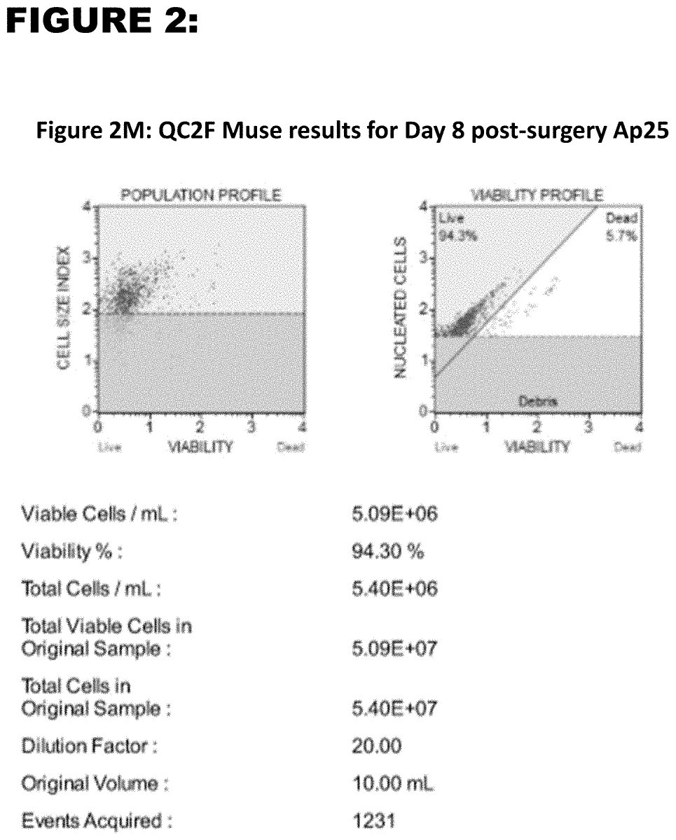

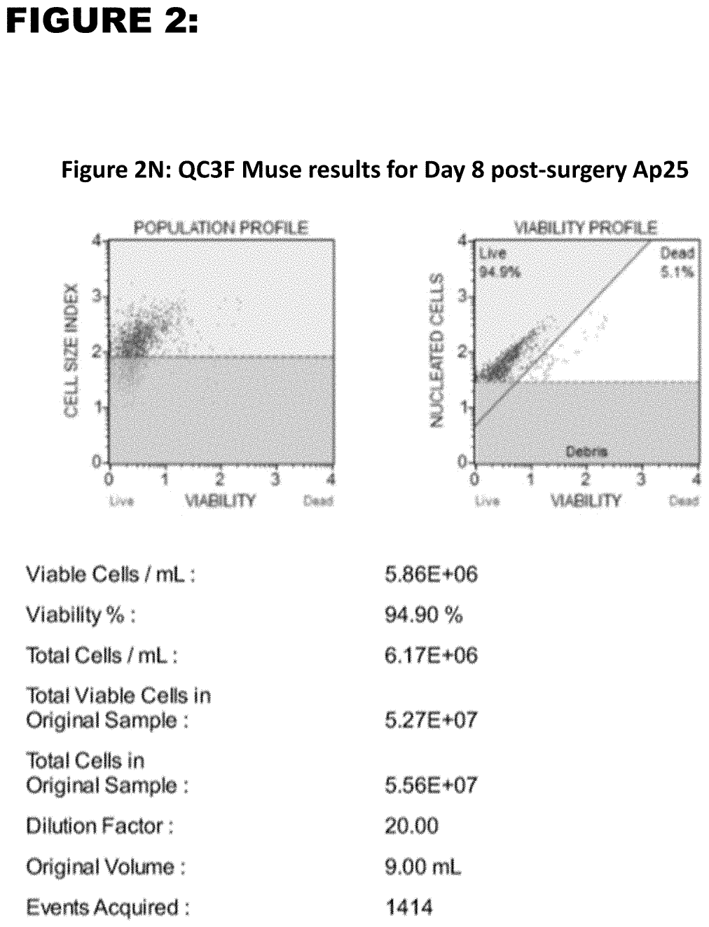

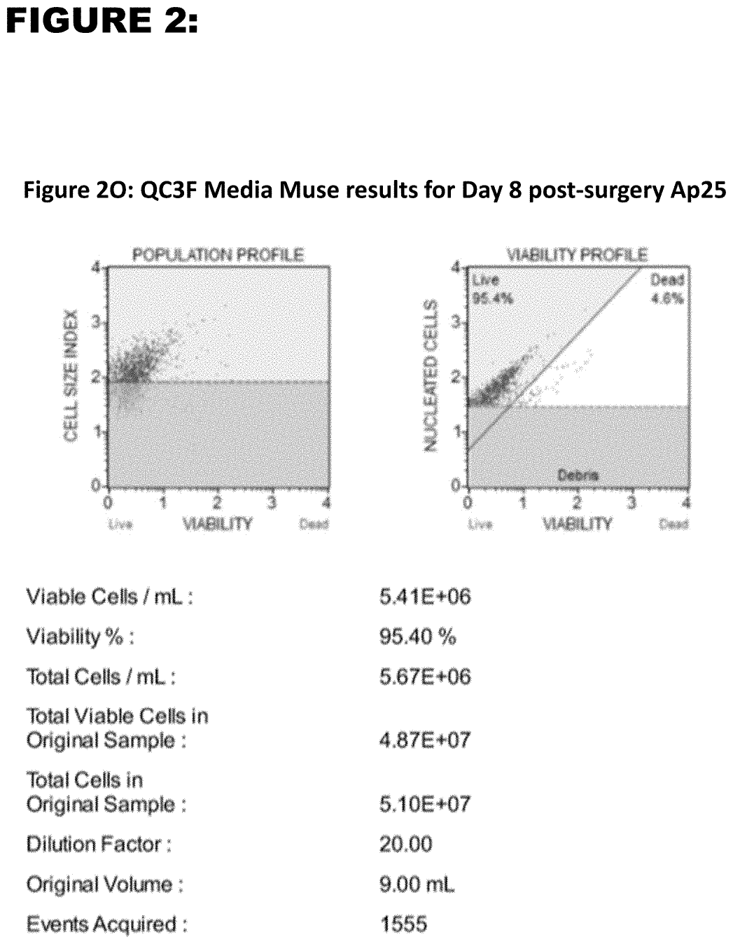

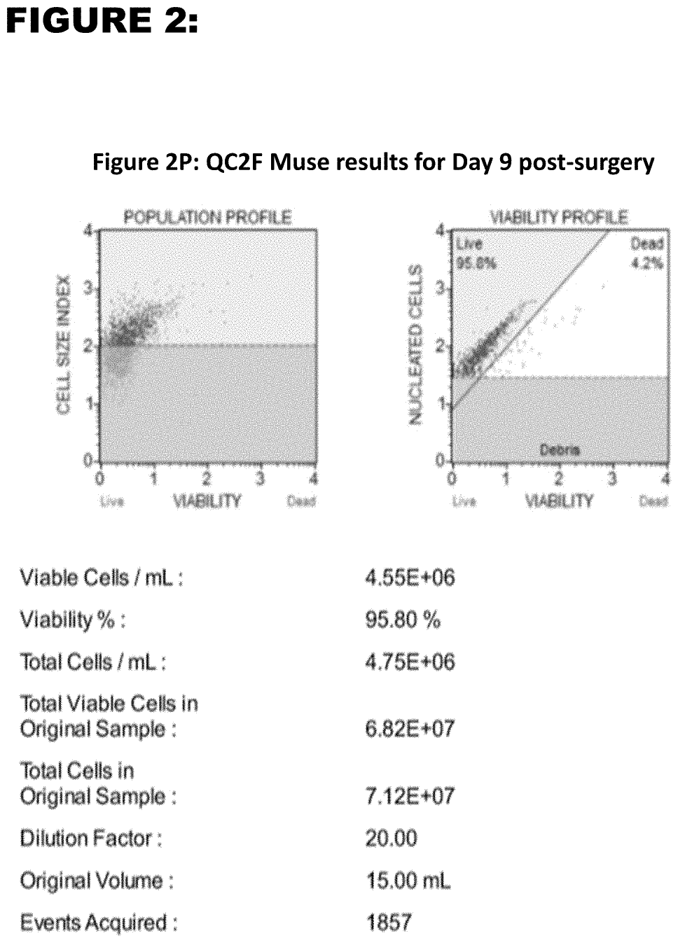

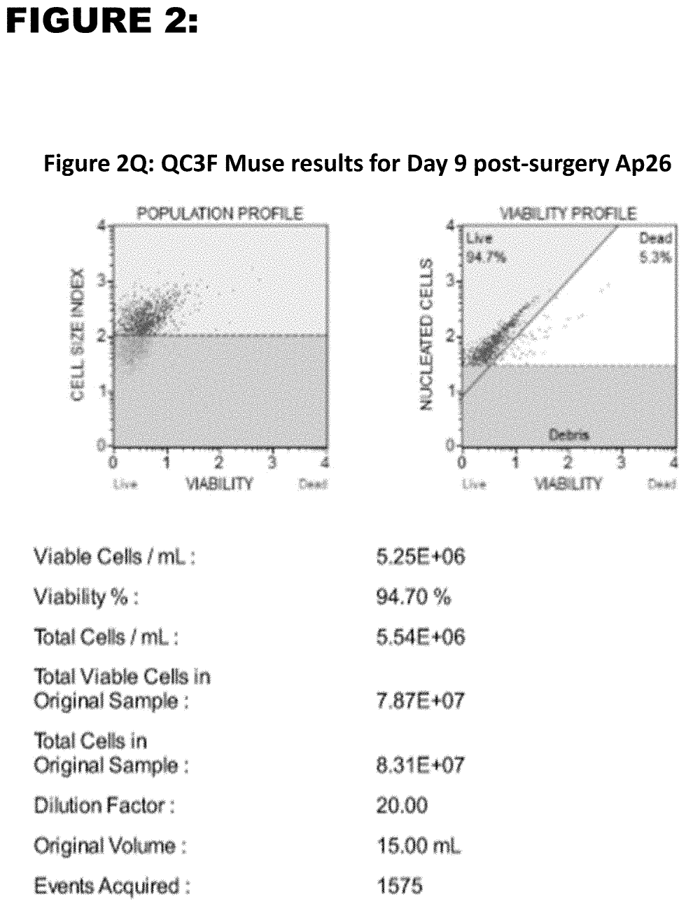

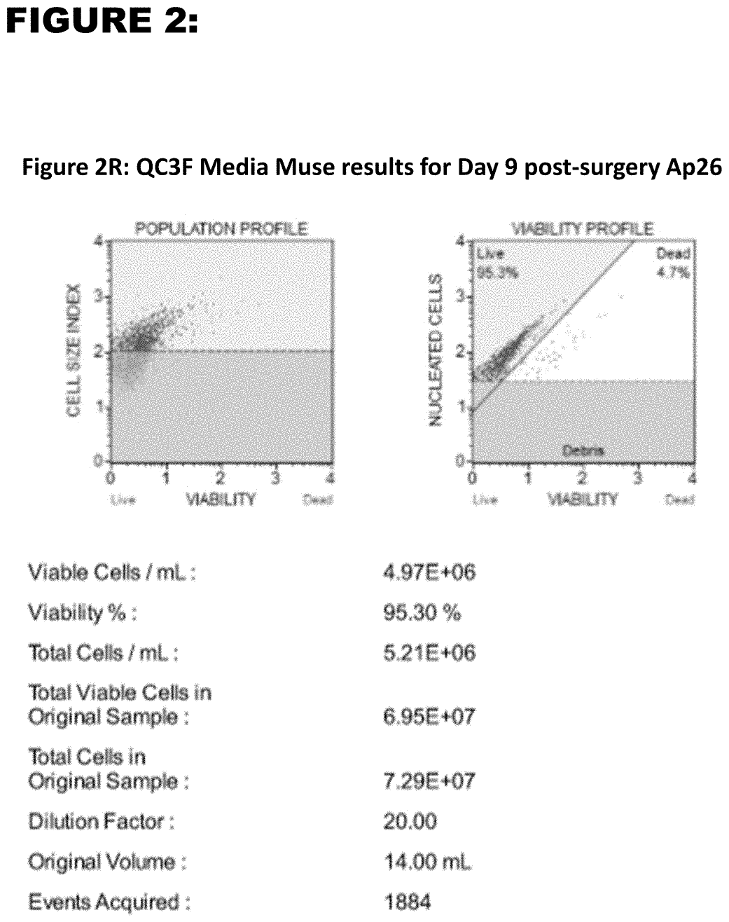

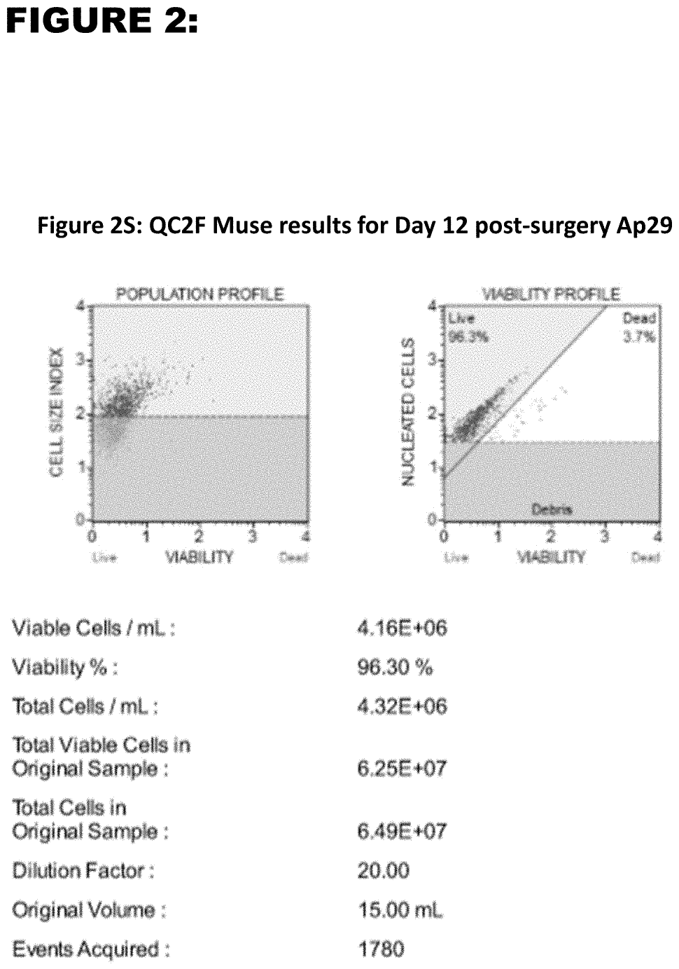

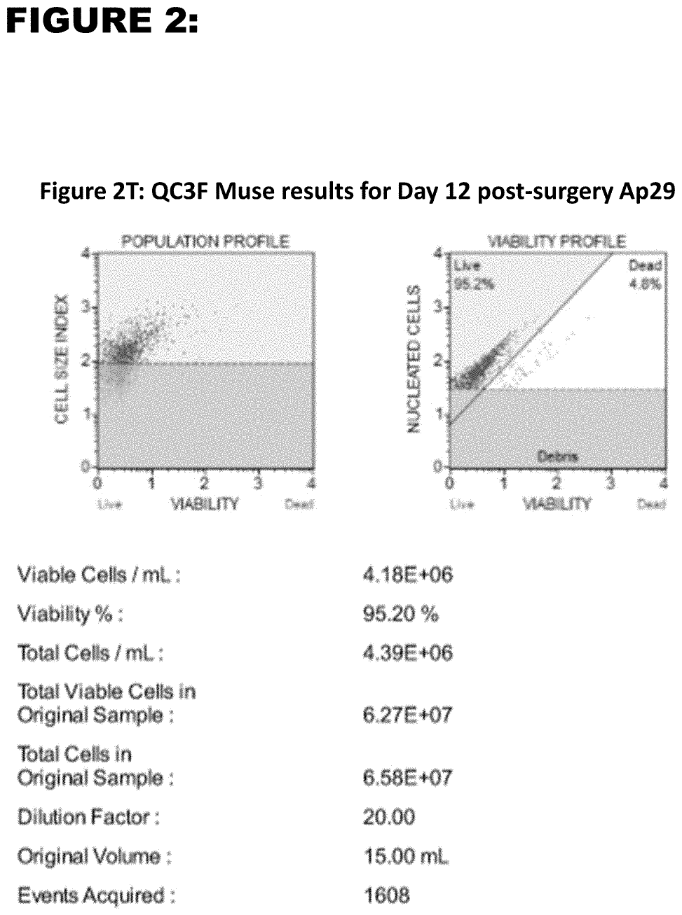

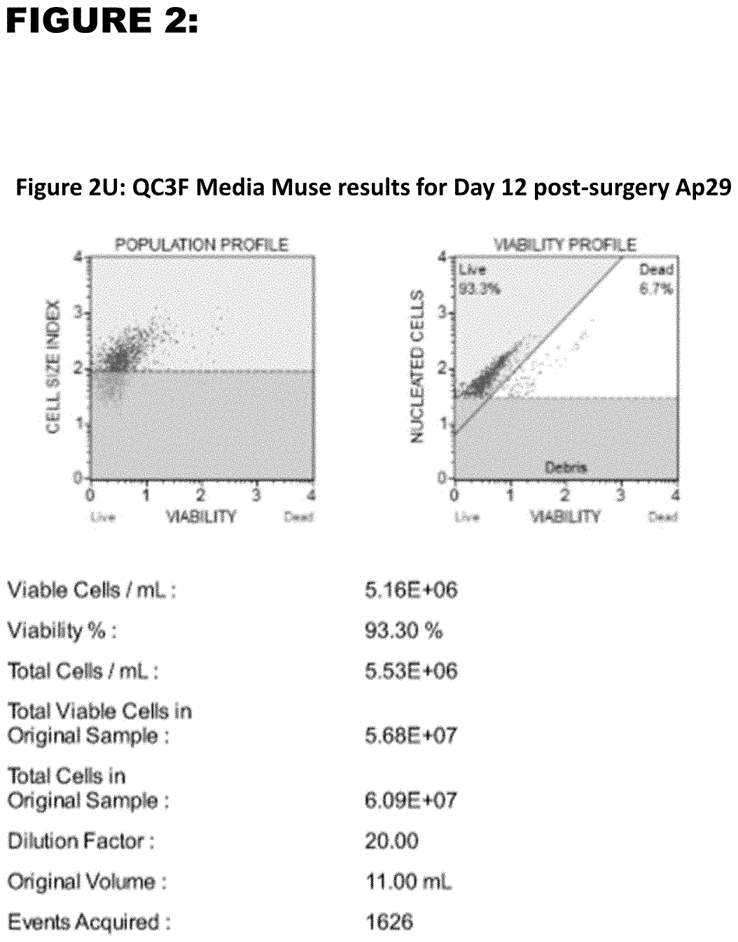

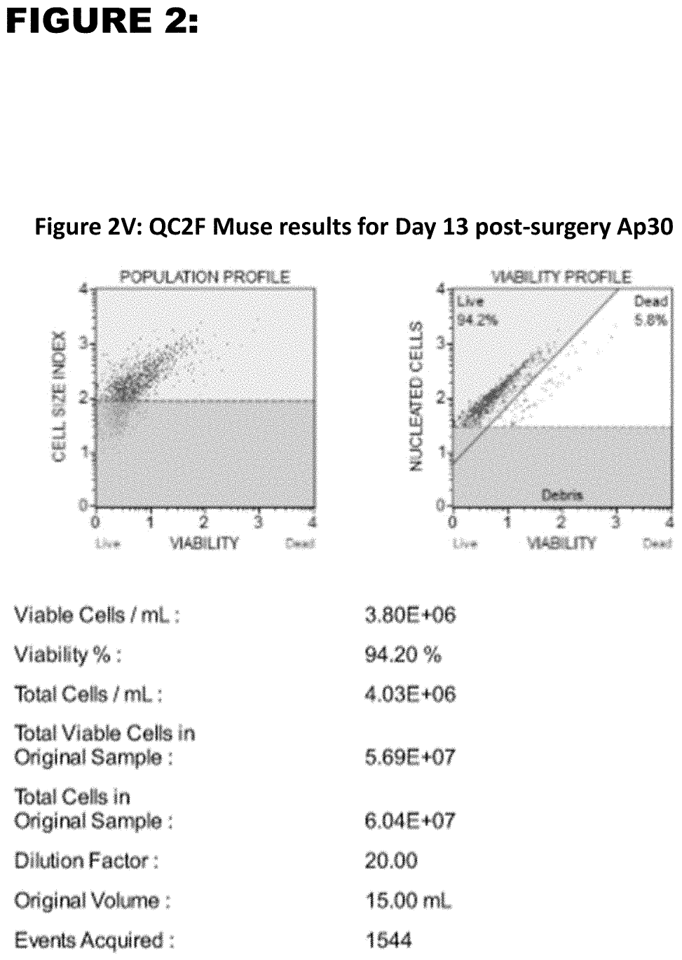

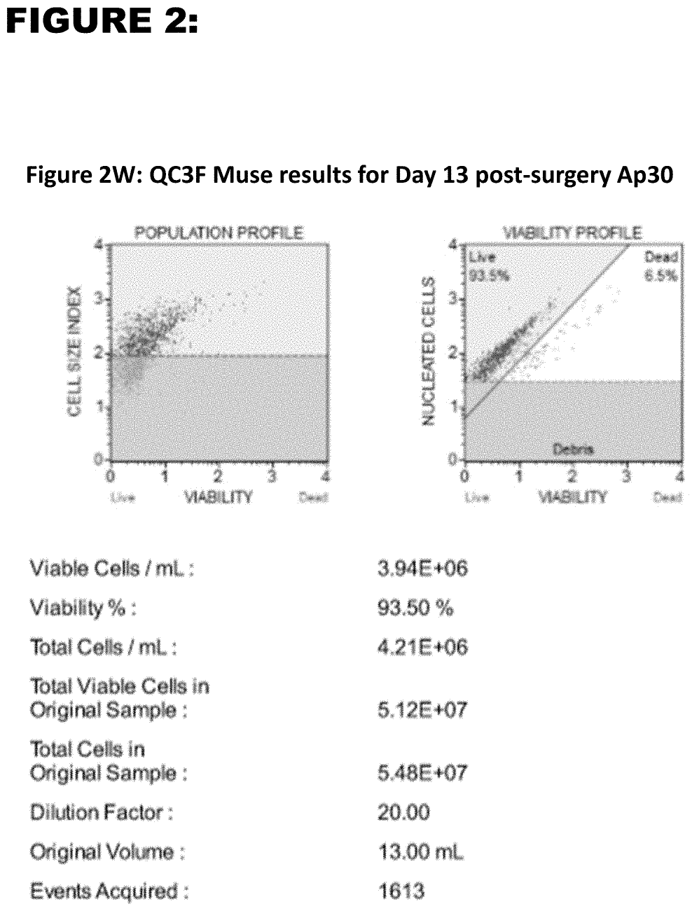

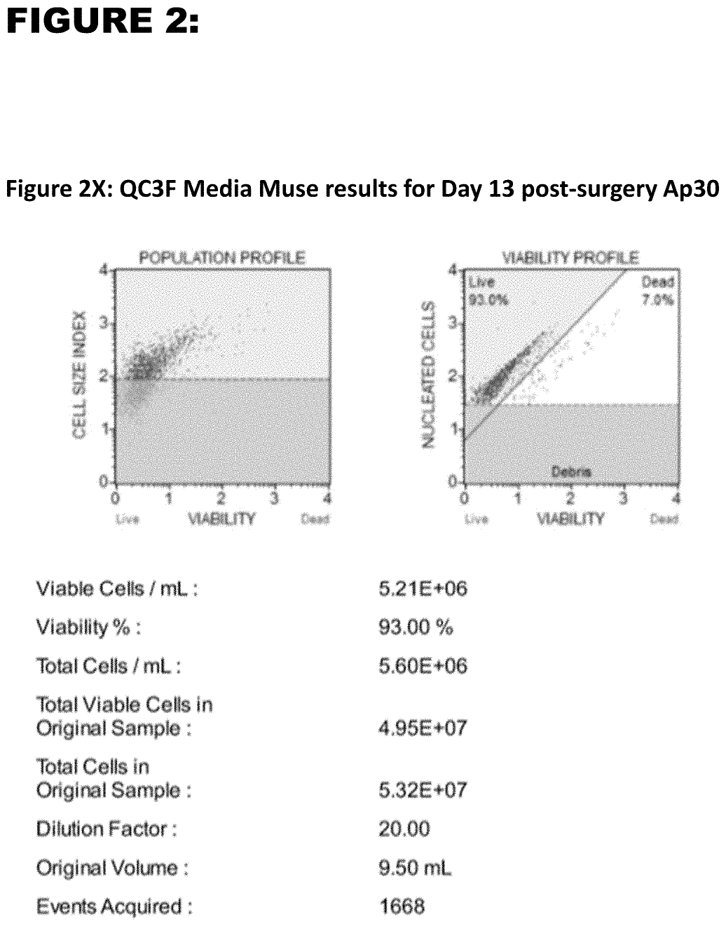

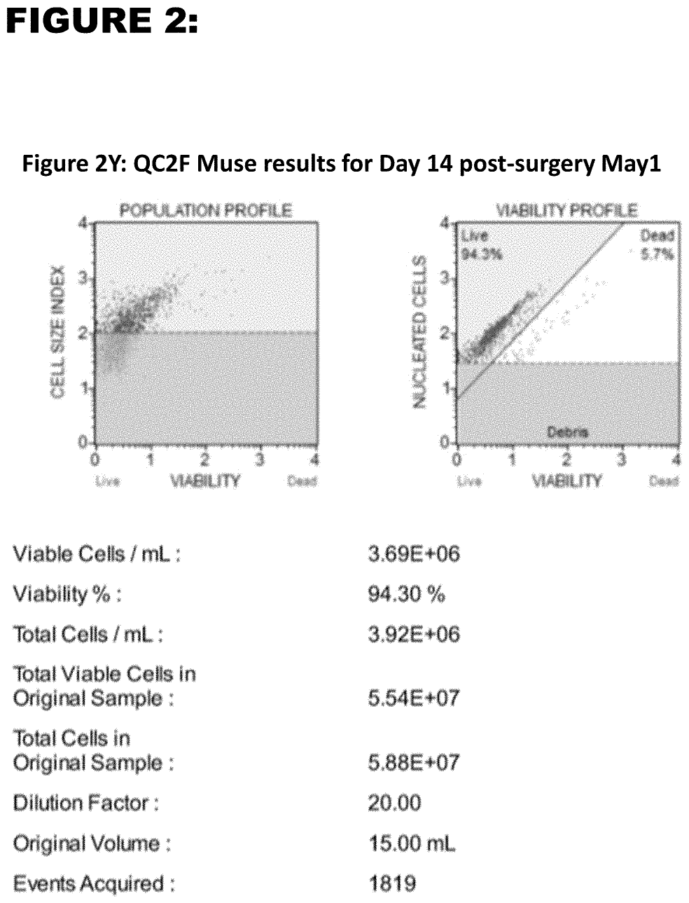

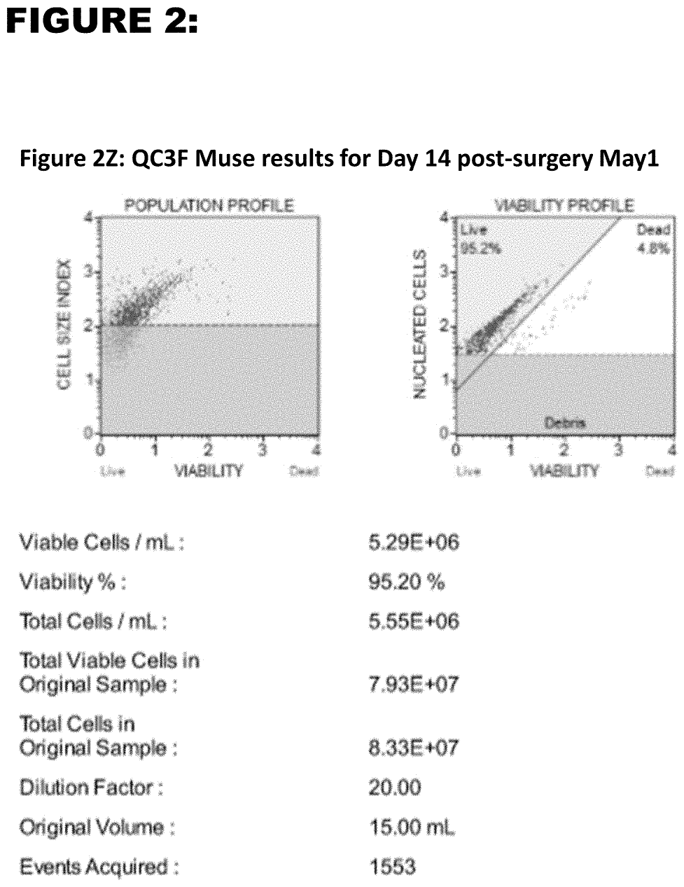

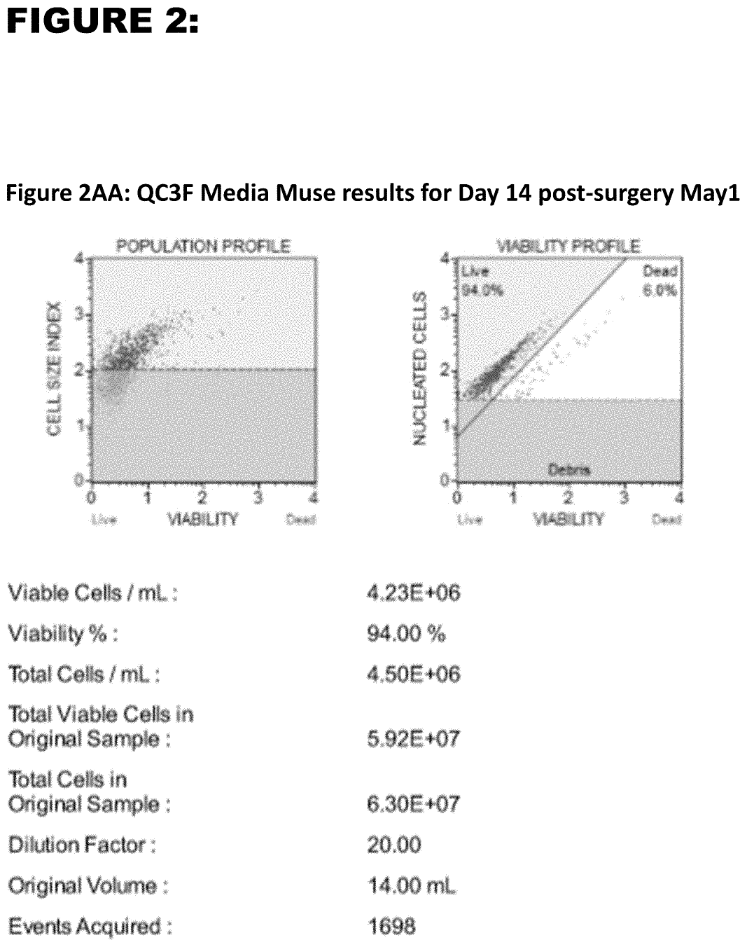

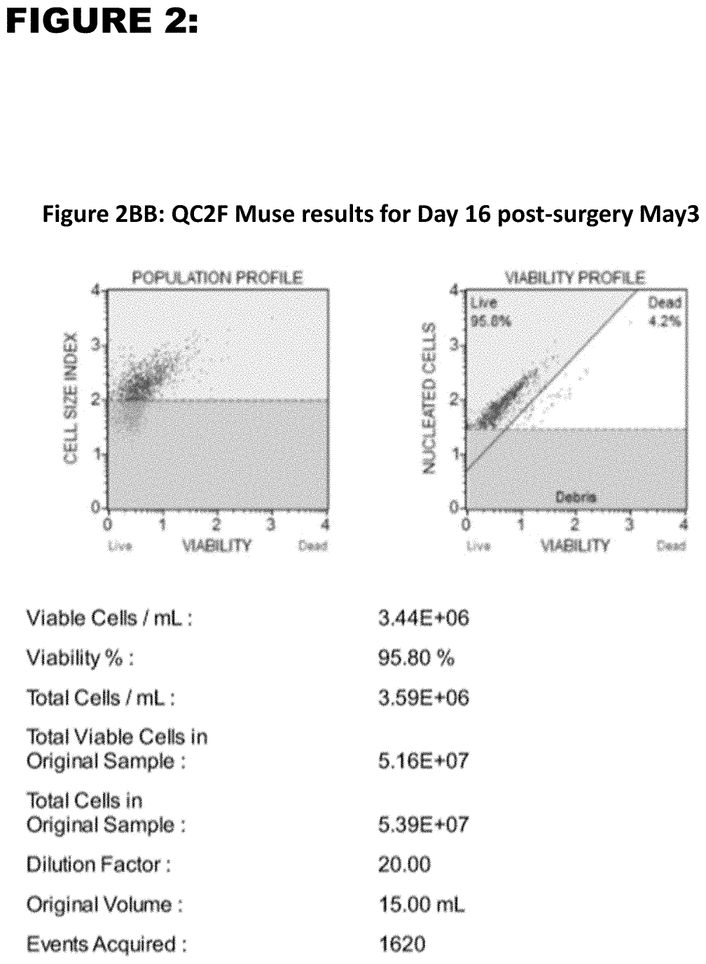

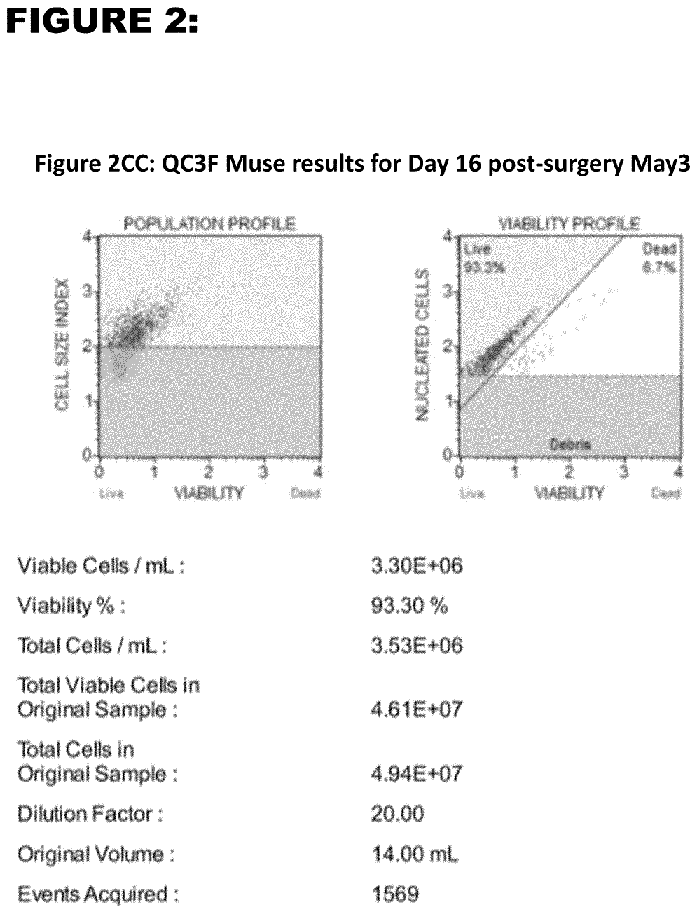

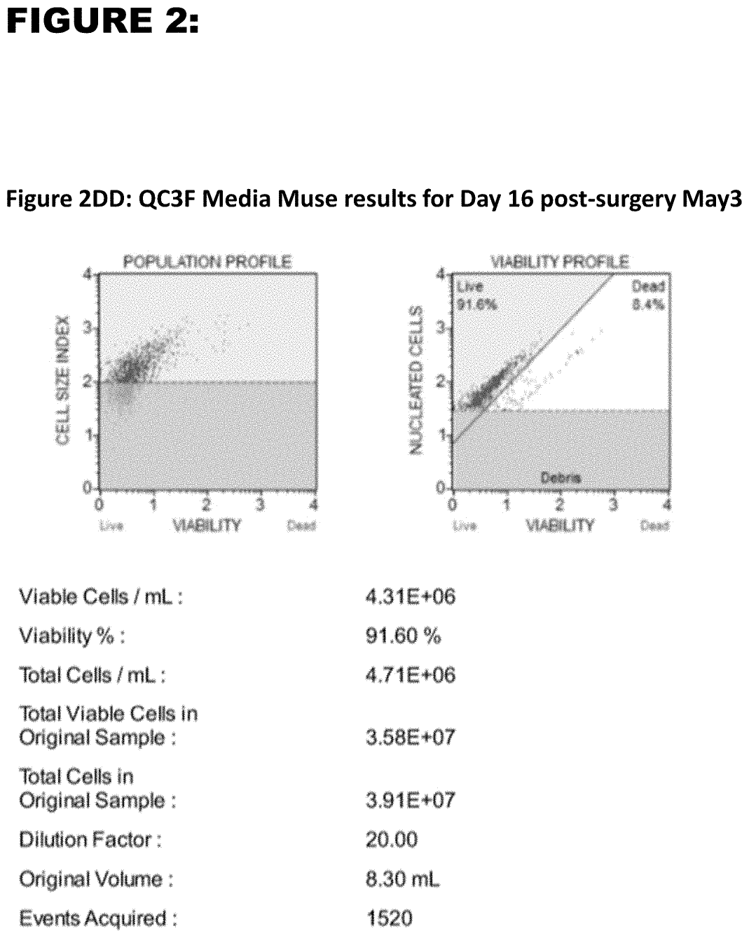

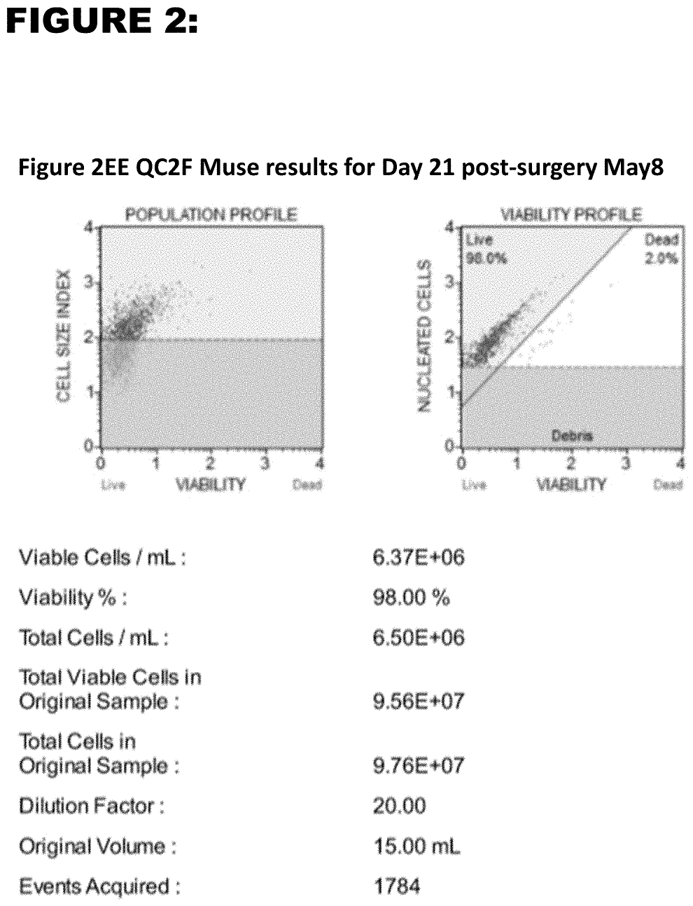

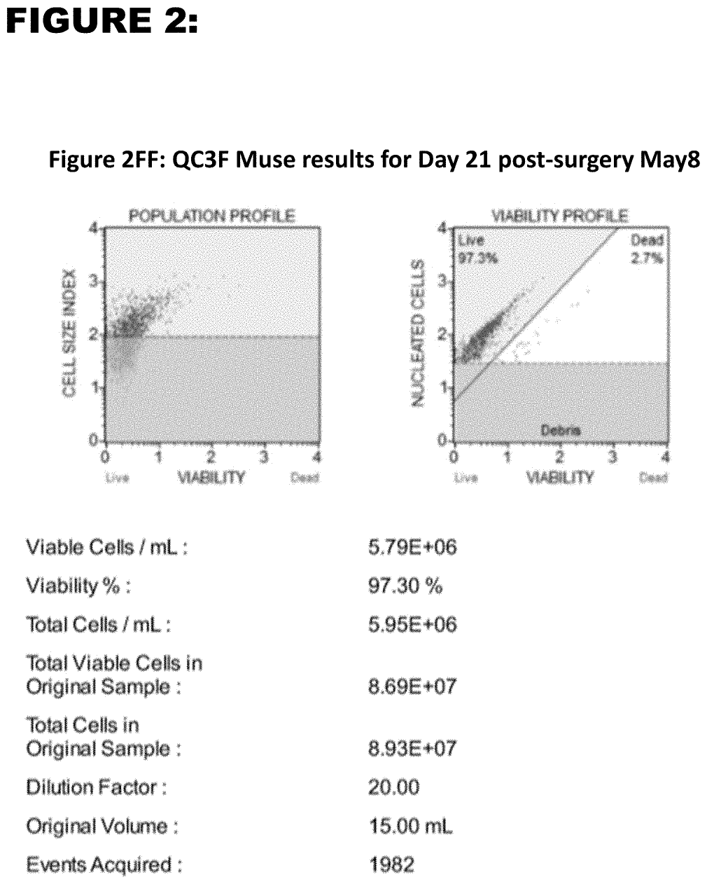

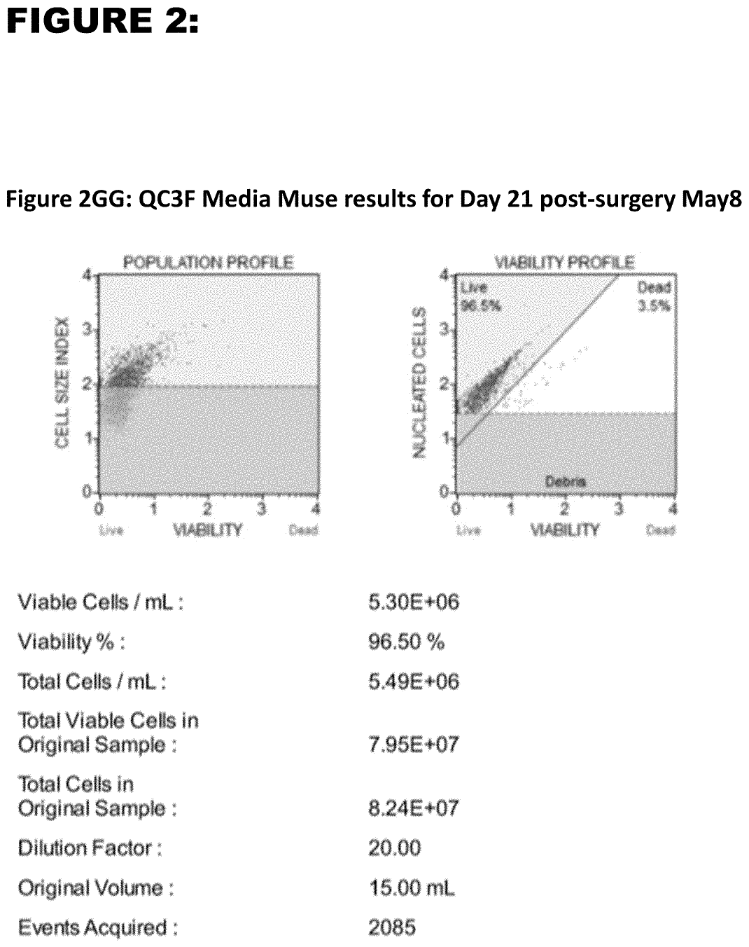

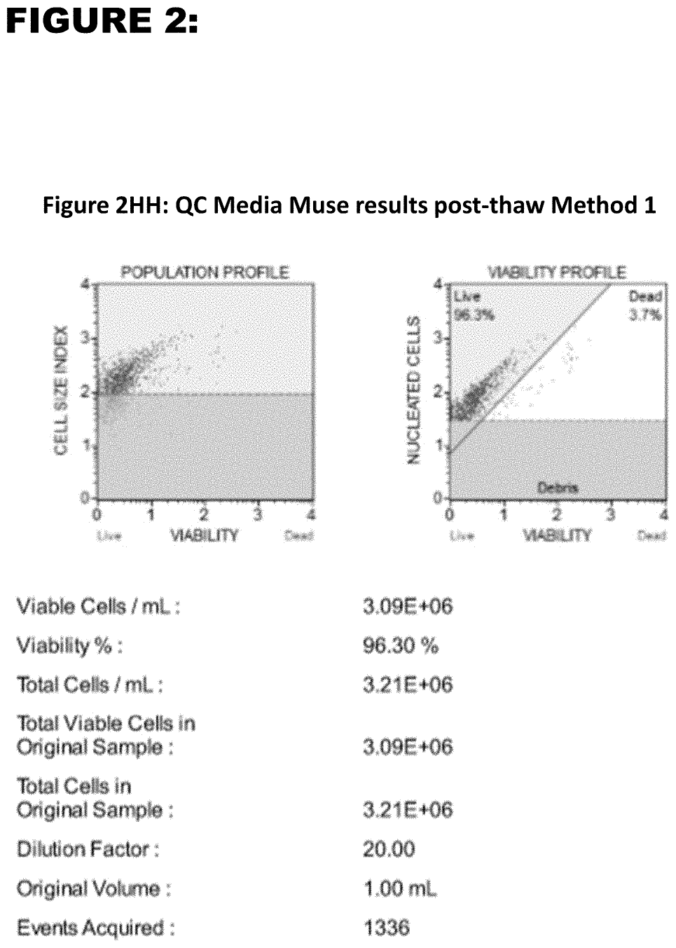

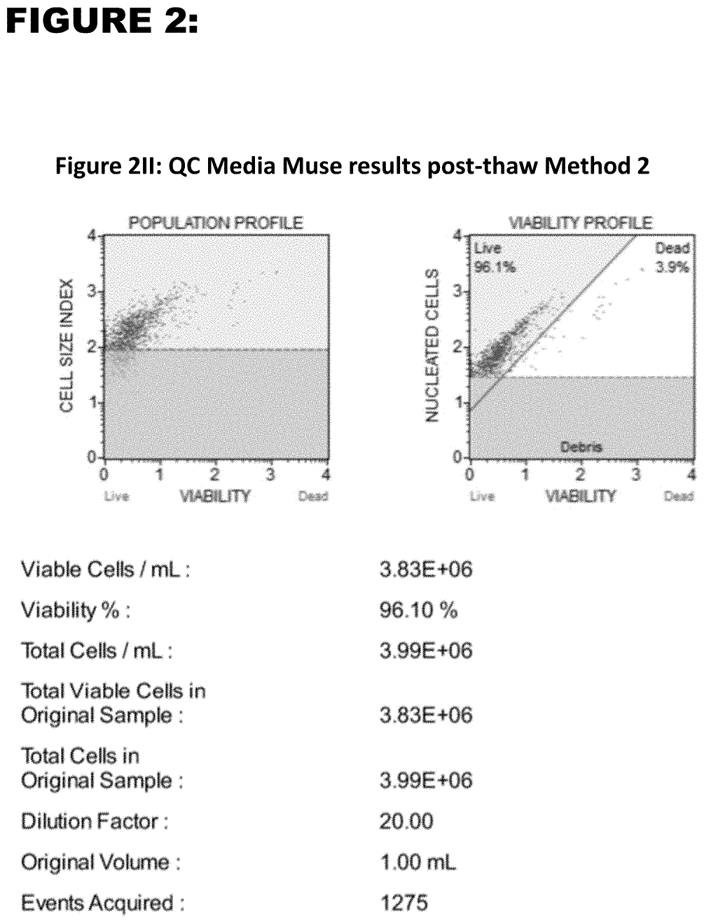

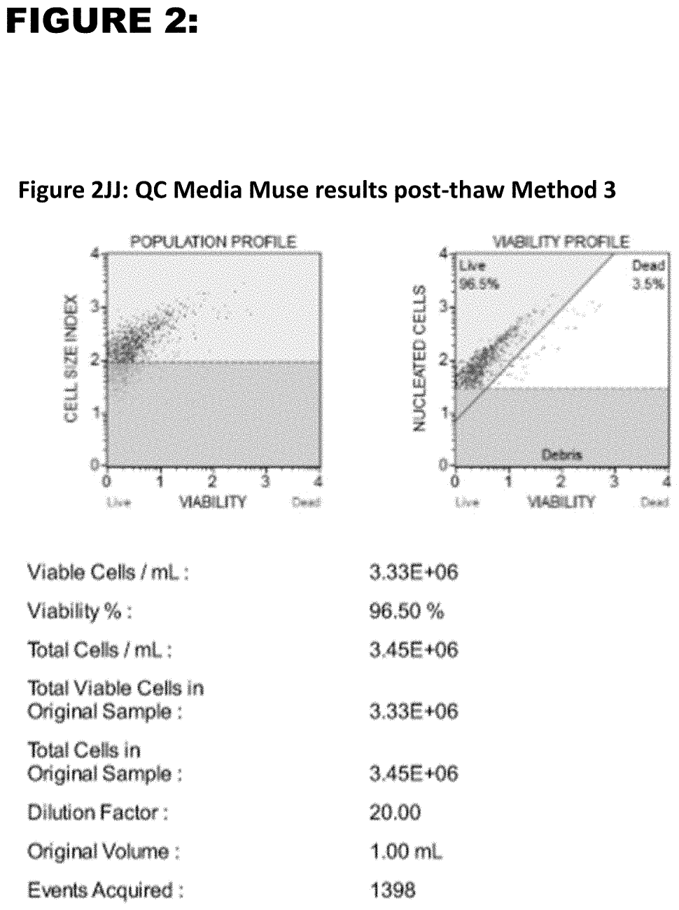

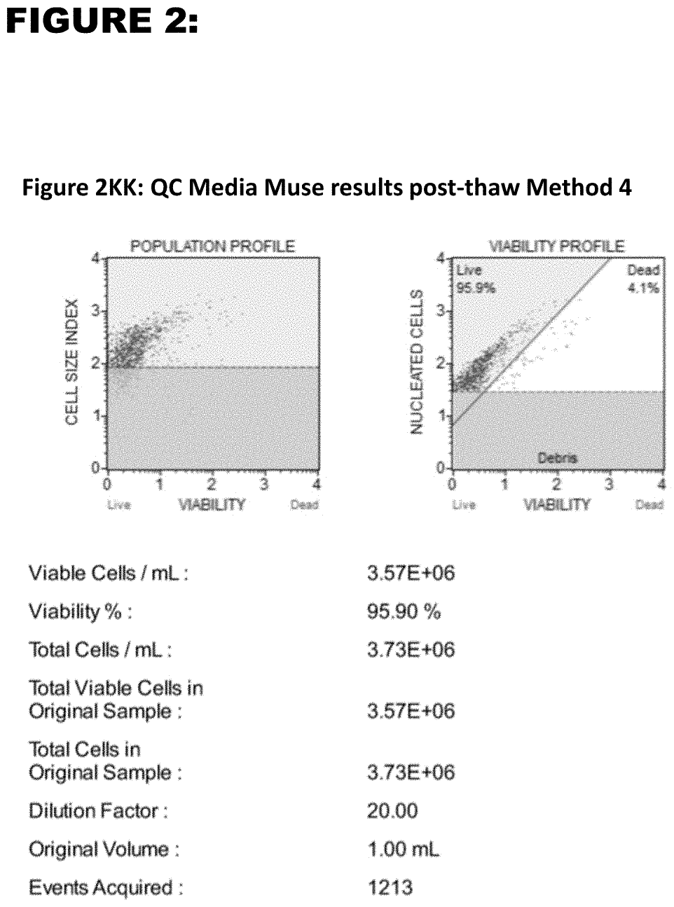

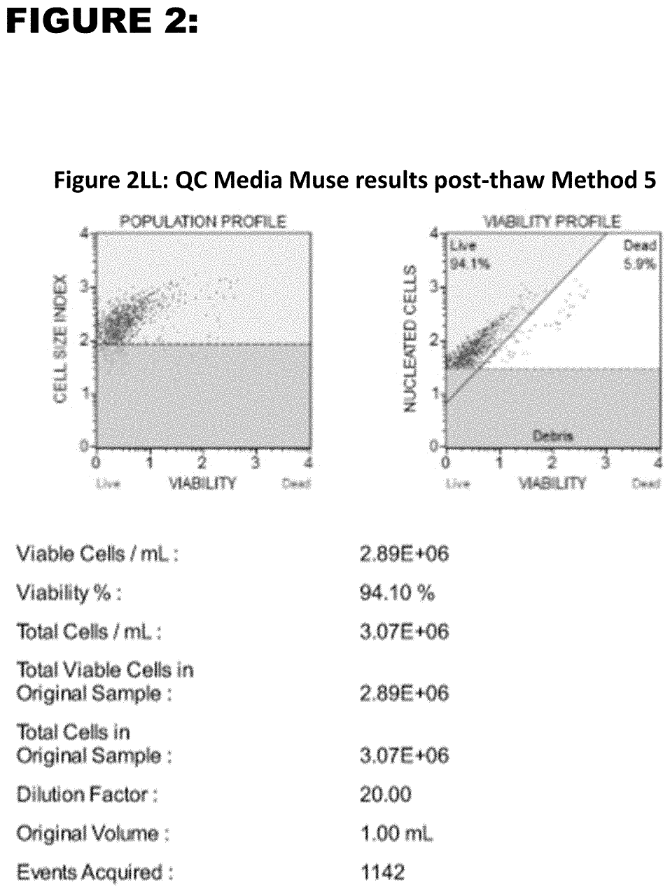

A lipoaspirate batch was aliquoted into 50 ml Falcon tubes and stored for several days. Stromal vascular fraction cell extraction was performed on days 1, 5, 6, 7, 8, 9, 12, 13, 14, 16, and 21 post-liposurgery. The Quality Control (QC) data is presented in Table 1 below and the MUSE graphic results are individually presented in FIGS. 2A-LL.

As shown in Table 1 and in the MUSE graphics results (FIGS. 2A-LL), the stromal vascular fraction cell extraction procedure offers a good method to isolate and enrich stromal vascular fraction cells with good viability and concentration.

Cryovials obtained from day 1 post-surgery were thawed. The cryovials were then vortexed for 10 seconds and QC was performed with MUSE by using a dilution 1/20. The results are presented in Table 2. The MUSE graphic results are also presented. The data shows that the enriched stromal vascular fraction maintains viability after cryopreservation and thawing.

TABLE 1
Aliquoted fat in Falcon tubes was stored in the refrigerator and the SVF cell extraction was performed at different days
post-liposurgery. The table shows the quality control (QC) data for the raw material tumescent (QC1T), QC for the processed
fat before filtering (QCF2), QC after filtering (QC3F), and QC for the final media added during the SVF pellet resuspension
(QC3F Media). Shaded cells represent the total cell/ml for the vials obtained per processed falcon tube.

| | Quality Control Fat | | | | | |
|---|---|---|---|---|---|---|
| | Day 8 post-surgery Ap25 | | | Day 9 post-surgery Ap26 | | |
| | | | QC3F | | | QC3F |
| | QC2F | QC3F | MEDIA | QC2F | QC3F | MEDIA |
| % Viability | 94.3 | 94.9 | 95.4 | 95.8 | 94.7 | 95.3 |
| Viable cells/ml | 5.09E+06 | 5.86E+06 | 5.41E+06 | 4.55E+06 | 5.25E+06 | 4.97E+06 |
| Total cells/ml | 5.40E+06 | 6.17E+06 | 5.67E+06 | 4.75E+06 | 5.54E+06 | 5.21E+06 |
| Total viable cells | 5.09E+07 | 5.27E+07 | 4.87E+06 | 6.82E+07 | 7.87E+07 | 6.95E+07 |
| in original sample | | | | | | |
| Total cells in | 5.40E+07 | 5.56E+07 | 5.10E+07 | 7.12E+07 | 8.31E+07 | 7.29E+07 |
| original sample | | | | | | |
| Number of | | | 9 | | | 14 |
| cryovials obtained | | | | | | |
| | Quality Control Fat | | | | | |
| | Day 12 post-surgery Ap29 | | | Day 13 post-surgery Ap30 | | |
| | QC2F | QC3F | QC2F | QC3F | QC2F | QC3F |
| % Viability | 96.3 | 95.2 | 96.3 | 95.2 | 96.3 | 95.2 |
| Viable cells/ml | 4.16E+06 | 4.18E+06 | 4.16E+06 | 4.18E+06 | 4.16E+06 | 4.18E+06 |
| Total cells/ml | 4.32E+06 | 4.39E+06 | 4.32E+06 | 4.39E+06 | 4.32E+06 | 4.39E+06 |
| Total viable cells | 6.25E+07 | 6.27E+07 | 6.25E+07 | 6.27E+07 | 6.25E+07 | 6.27E+07 |
| in original sample | | | | | | |
| Total cells in | 6.49E+07 | 6.58E+07 | 6.49E+07 | 6.58E+07 | 6.49E+07 | 6.58E+07 |
| original sample | | | | | | |
| Number of | | | | | | |
| cryovials obtained | | | | | | |

| | Quality Control Fat | | | | | | | | |
|---|---|---|---|---|---|---|---|---|---|
| | Day 14 post-surgery May1 | | | Day 16 post-surgery May3 | | | Day 21 post-surgery May8 | | |
| | | | QC3F | | | QC3F | | | QC3F |
| | QC2F | QC3F | MEDIA | QC2F | QC3F | MEDIA | QC2F | QC3F | MEDIA |
| % Viability | 94.3 | 95.2 | 94 | 95.8 | 93.3 | 91.60 | 98 | 97.3 | 96.5 |
| Viable cells/ml | 3.69E+06 | 5.29E+06 | 4.23E+06 | 3.44E+06 | 3.30E+06 | 4.31E+06 | 6.37E+06 | 5.79E+06 | 5.30E+06 |
| Total cells/ml | 3.92E+06 | 5.55E+06 | 4.50E+06 | 3.59E+06 | 3.53E+06 | 4.71E+06 | 6.50E+06 | 5.95E+06 | 5.49E+06 |
| Total viable cells | 5.54E+07 | 7.93E+07 | 5.92E+07 | 5.16E+07 | 4.61E+07 | 3.58E+07 | 9.56E+07 | 8.69E+07 | 7.95E+07 |
| in original sample | | | | | | | | | |
| Total cells in | 5.88E+07 | 8.33E+07 | 6.30E+07 | 5.39E+07 | 4.94E+07 | 3.91E+07 | 9.76E+07 | 8.93E+07 | 8.24E+07 |
| original sample | | | | | | | | | |
| Number of | | | 14 | | | 8 | | | 14 |
| cryovials obtained | | | | | | | | | |

TABLE 2
Quality Control results of Batch AC10004171901001, Day
1 post-surgery following different thawing methods and
its comparison with pre-freezing quality control data.

| | | Batch AC10004171901001 | |
|---|---|---|---|
| | | Day 1 post-surgery Ap18 | |
| | | QC3F MEDIA | QC3F MEDIA |
| Method | Analysis | BEFORE FREEZING | POST FREEZING |
| 1 | % Viability: | | 96.3 |
| | Viable cells/ml: | | 3.09E+06 |
| | Total cells/ml: | | 3.21E+06 |
| 2 | % Viability: | | 96 |
| | Viable cells/ml: | | 3.83E+06 |
| | Total cells/ml: | | 3.99E+06 |
| 3 | % Viability: | 95.8 | 96.5 |
| | Viable cells/ml: | 3.88E+06 | 3.33E+06 |
| | Total cells/ml: | 4.05E+06 | 3.45E+06 |
| 4 | % Viability: | | 95 |
| | Viable cells/ml: | | 3.57E+06 |
| | Total cells/ml: | | 3.73E+06 |
| 5 | % Viability: | | 94 |
| | Viable cells/ml: | | 2.89E+06 |
| | Total cells/ml: | | 3.07E+06 |

### Example 4: Lipoaspirate Fractions

A lipoaspirate may be separated into tumescent and fat fractions by allowing the lipoaspirate to rest for least 10 min at room temperature. The tumescent fraction and fat fraction may then be processed separately as indicated above to result in an adipose tissue stromal vascular fraction. Such fractions may be stored separately, and may be indicated, for instance, by different color vials (i.e., one color for tumescent fraction and another color for the fat fraction). Viability may be evaluated in each fraction at several different time points. For instance, total cell viability may be measured before the adipose tissue is processed (e.g. the raw adipose tissue), before filtering, after filtering, before cryopreservation, or after cryopreservation for each of the tumescent and fat fractions.

1. A composition, the composition comprising an adipose tissue stromal vascular fraction, wherein the adipose tissue stromal vascular fraction
(a) is derived from adipose tissue that meets the following criteria:
1) the adipose tissue is collected from a subject between 18 and 45 years of age,
2) the adipose tissue is collected from a visceral fat source, and
3) the adipose tissue is collected from a subject with a BMI less than 30;
(b) comprises about 4.2 to about 9.2×10⁷ nucleated cells per 100 ml of adipose tissue collected, wherein at least about 85% of the nucleated cells express CD90 on their cell surface; and
(c) comprises mesenchymal stem cells, wherein the mesenchymal stem cells are about 95 to about 99% viable as measured within forty-eight (48) hours of collection, and do not display detectable culture-associated cellular changes.

2. The composition of claim 1, wherein the adipose tissue stromal vascular fraction is derived from adipose tissue collected from a subject with no clinically significant abnormalities in a physical exam administered within six (6) months prior to taking the adipose tissue.

3.-6. (canceled)

7. The composition of claim 1, wherein the adipose tissue is collected from a subject with no known history of lipoma/lipomatosis, no known history of cancer in the last five years, no known history of lymphoma, leukemia or Kaposi sarcoma, and no known history of diabetes.

8. The composition of claim 1, wherein the adipose tissue stromal vascular fraction comprises about 5.0 to about 9.2×10⁷ nucleated cells per 100 ml of adipose tissue collected.

9-12. (canceled)

13. The composition of claim 1, wherein at least 70% of the cells expressing CD90 also express CD29.

14. The composition of claim 1, wherein CD34 expression was detected in less than 1% of cells in the adipose tissue stromal vascular fraction.

15. The composition of claim 1, wherein the nucleated cells did not express detectable levels of CD45 or CD31 by FACS analysis.

16.-31. (canceled)

32. A composition, the composition comprising an adipose tissue stromal vascular fraction, wherein the adipose tissue stromal vascular fraction
(a) is derived from a tumescent fraction of a lipoaspirate that meets the following criteria:
1) the lipoaspirate is collected from a subject between 18 and 45 years of age,
2) the lipoaspirate is collected from a visceral fat source, and
3) the lipoaspirate is collected from a subject with a BMI less than 30;
(b) comprises about 4.2 to about 9.2×10⁶ nucleated cells per 100 ml of lipoaspirate collected, wherein at least about 85% of the nucleated cells express CD90 on their cell surface; and
(c) comprises mesenchymal stem cells, wherein the mesenchymal stem cells are about 95 to about 99% viable as measured within forty-eight (48) hours of collection, and do not display detectable culture-associated cellular changes.

33. The composition of claim 32, wherein the adipose tissue stromal vascular fraction is derived from a tumescent fraction of a lipoaspirate collected from a subject with no clinically significant abnormalities in a physical exam administered within 6 months prior to taking the lipoaspirate.

34.-37. (canceled)

38. The composition of claim 32, wherein the adipose tissue is collected from a subject with no known history of lipoma/lipomatosis, no known history of cancer in the last five years, no known history of lymphoma, leukemia or Kaposi sarcoma, and no known history of diabetes.

39. The composition of claim 32, wherein the adipose tissue stromal vascular fraction comprises about 5.0 to about 9.2×10⁶ nucleated cells per 100 ml of tumescent fraction of a lipoaspirate collected.

40-43. (canceled)

44. The composition of claim 32, wherein at least 70% of the cells expressing CD90 also express CD29.

45. The composition of claim 32, wherein CD34 expression was detected in less than 1% of cells in the adipose tissue stromal vascular fraction.

46. The composition of claim 32, wherein the nucleated cells did not express detectable levels of CD45 or CD31 by FACS analysis.

47.-55. (canceled)

56. A composition, the composition comprising an adipose tissue stromal vascular fraction, wherein the adipose tissue stromal vascular fraction
(a) is derived from a fat fraction of a lipoaspirate that meets the following criteria:
1) the lipoaspirate is collected from a subject between 18 and 45 years of age,
2) the lipoaspirate is collected from a visceral fat source, and
3) the lipoaspirate is collected from a subject with a BMI less than 30;
(b) comprises about 4.2 to about 9.2×10⁷ nucleated cells per 100 ml of lipoaspirate collected, wherein at least about 85% of the nucleated cells express CD90 on their cell surface; and
(c) comprises mesenchymal stem cells, wherein the mesenchymal stem cells are about 95 to about 99% viable as measured within forty-eight (48) hours of collection, and do not display detectable culture-associated cellular changes.

57. The composition of claim 56, wherein the adipose tissue stromal vascular fraction is derived from a fat fraction of a lipoaspirate collected from a subject with no clinically significant abnormalities in a physical exam administered within 6 months prior to taking the lipoaspirate.

58.-61. (canceled)

62. The composition of claim 56, wherein the a fat fraction of a lipoaspirate is collected from a subject with no known history of lipoma/lipomatosis, no known history of cancer in the last five years, no known history of lymphoma, leukemia or Kaposi sarcoma, and no known history of diabetes.

63. The composition of claim 56, wherein the adipose tissue stromal vascular fraction comprises about 5.0 to about 9.2×10⁷ nucleated cells per 100 ml of fat fraction of a lipoaspirate collected.

64-67. (canceled)

68. The composition of claim 56, wherein at least 70% of the cells expressing CD90 also express CD29.

69. The composition of claim 56, wherein CD34 expression was detected in less than 1% of cells in the adipose tissue stromal vascular fraction.

70. The composition of claim 56, wherein the nucleated cells did not express detectable levels of CD45 or CD31 by FACS analysis.

71-81. (canceled)
